SEQUENCE LISTING (15 sequences)

SEQ ID NO: 1 - protein, 698 aa, Artificial Sequence
  Protein 1
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 2 - protein, 733 aa, Artificial Sequence
  Protein 2
SMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 3 - protein, 698 aa, Artificial Sequence; identical to SEQ ID NO: 1
  Protein 3

SEQ ID NO: 4 - protein, 660 aa, Artificial Sequence
  Protein 4
GGVPGAVPGGVPGGVFYPGAGFGAVPGGVADAAAAYKAAKAGAGLGGVPGVGGLGVSAGAVVPQPGAGVKPGKVPGVGLPGVYPGFGAVPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGFGAVPGVGGAGVPGVPGAIPGIGGIAGVGTPAAAAAAAAAAKAAKYGAAAGLVPGGPGFGPGVVGVPGFGAVPGVGVPGAGIPVVPGAGIPGAAGFGAVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 5 - protein, 732 aa, Artificial Sequence
  Protein 5
SMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 6 - protein, 698 aa, Artificial Sequence; identical to SEQ ID NO: 1
  Protein 6

SEQ ID NO: 7 - protein, 660 aa, Artificial Sequence
  Protein 7
MGGVPGAVPGGVPGGVFYPGAGFGAVPGGVADAAAAYKAAKAGAGLGGVPGVGGLGVSAGAVVPQPGAGVKPGKVPGVGLPGVYPGFGAVPGARFPGVGVLPGVPTGAGVKPKAPGVGGAFAGIPGVGPFGGPQPGVPLGYPIKAPKLPGGYGLPYTTGKLPYGYGPGGVAAAGKAGYPTGTGVGPQAAAAAAAKAAAKFGAGAAGFGAVPGVGGAGVPGVPGAIPGIGGIAGVGTPAAAAAAAAAAKAAKYGAAAGLVPGGPGFGPGVVGVPGFGAVPGVGVPGAGIPVVPGAGIPGAAGFGAVSPEAAAKAAAKAAKYGARPGVGVGGIPTYGVGAGFFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 8 - protein, 147 aa, Artificial Sequence
  Protein 8
SAMGGVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 9 - protein, 200 aa, Artificial Sequence
  Protein 9
SAMGALVGLGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 10 - protein, 56 aa, Artificial Sequence
  Protein 10
GIPPAAAAKAAKYGAAGLGGVLGGAGQFPLGGVAARPGFGLSPIFPGGACLGKACG

SEQ ID NO: 11 - protein, 47 aa, Artificial Sequence
  Protein 11
GAAGLGGVLGGAGQFPLGGVAARPGFGLSPIFPGGACLGKACGRKRK

SEQ ID NO: 12 - protein, 34 aa, Artificial Sequence
  Protein 12
GADEGVRRSLSPELREGDPSSSQHLPSTPSSPRV

SEQ ID NO: 13 - protein, 34 aa, Artificial Sequence
  Protein 13
GADEGVRRSLSPELREGDPSSSQHLPSTPSSPRF

SEQ ID NO: 14 - protein, 216 aa, Artificial Sequence
  Protein 14
AAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 15 - protein, 183 aa, Artificial Sequence
  Protein 15
AAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

(19) United States
(12) Patent Application Publication
(10) Pub. No.: US 2023/0108501 A1
(43) Pub. Date: Apr. 6, 2023

(54) TISSUE ENGINEERING SCAFFOLDS

(71) Applicant: ALLERGAN PHARMACEUTICALS INTERNATIONAL LIMITED, Dublin (IE)

(72) Inventors: Anthony Steven WEISS, Sydney (AU); Behnaz AGHAEI-GHAREH-BOLAGH, Sydney (AU)

(73) Assignee: ALLERGAN PHARMACEUTICALS INTERNATIONAL LIMITED, Dublin (IE)

(21) Appl. No.: 17/798,065

(22) PCT Filed: Feb. 8, 2021
(86) PCT No.: PCT/IB2021/051014

§ 371 Date: Aug. 5, 2022

Related U.S. Application Data
(60) Provisional application No. 62/971,195, filed on Feb. 6, 2020.
(60) Provisional application No. 63/022,253, filed on May 8, 2020.

(51) Int. Cl. A61L 27/26; A61L 27/58

(52) U.S. Cl. CPC A61L 27/26; A61L 27/58; A61L 2430/04; A61L 2430/22; A61L 2430/34

(57) ABSTRACT

The present disclosure relates to a hybrid material, such as a hybrid yarn, as well as methods of making and using the same. The hybrid material may include tropoelastin. Further, the hybrid material can also include a biodegradable polymer. In addition, the disclosure is also directed to compositions and methods for treating a tissue, such as treatment of organ prolapse.

### CROSS-REFERENCE TO RELATED APPLICATIONS

This application claims the benefit of and priority to U.S. Provisional Application No. 62/971,195, filed Feb. 6, 2020, and U.S. Provisional Application No. 63/022,253, filed May 8, 2020, the entire contents of each of which are hereby incorporated herein by reference in their entirety.

### FIELD

Methods of making scaffolds comprising tropoelastin are described. Methods for reconstruction of the body using tissue engineering scaffolds are also contemplated. These methods include the steps of providing a tissue engineering scaffold comprising the tropoelastin and a synthetic polymer to an area of tissue. Methods of treating organ prolapse are also considered.

### BACKGROUND

Pelvic organ prolapse is a condition that may affect women. Non-degradable synthetic meshes are used for the transvaginal surgical repair of pelvic organ prolapse. However, use of current synthetic meshes is associated with frequent adverse events, such as tissue erosion, leading to bans by regulatory authorities in many countries. Thus, there is an unmet demand for elastic, implantable, biologically compatible meshes.

Elastin is a protein component of the ECM and provides elasticity to tissues throughout the body. Tropoelastin, the monomer subunit of elastin, has been used with success in electrospun scaffolds as it is a naturally cell interactive polymer. Scaffolds that incorporate tropoelastin support cell attachment and proliferation, and have been proven to encourage elastogenesis and angiogenesis in vitro and in vivo.

Tropoelastin has been previously linked to tissue repair and wound healing. However, there is an unmet need for improved tropoelastin tissue engineering scaffolds that promote tissue repair by enabling cell attachment and proliferation. The disclosure addresses that need, providing methods and compositions comprising biocompatible, biodegradable, and non-toxic scaffolds with mechanical properties similar to the native tissue of the intended implant site.

### SUMMARY

In a first aspect a method of making a hybrid material is provided. The method comprises: providing tropoelastin, providing a biodegradable polymer, and mixing the tropoelastin and biodegradable polymer to produce a mixture; wherein the mixture results in a hybrid material.

In some embodiments of any of the below- or above-mentioned embodiments, the method further comprises melting the biodegradable polymer after the providing step, thereby producing a molten biodegradable polymer, and suspending the tropoelastin in the molten biodegradable polymer prior to the mixing step.

In some embodiments of any of the below- or above-mentioned embodiments, the tropoelastin is provided as a monomer in solution. In some embodiments of any of the below- or above-mentioned embodiments, the tropoelastin is provided as tropoelastin particles.

In some embodiments of any of the below- or above-mentioned embodiments, the method further comprises dissolving the biodegradable polymer and dissolving the tropoelastin prior to the mixing step and mixing the dissolved biodegradable polymer and the dissolved tropoelastin.

In some embodiments of any of the below- or above-mentioned embodiments, the method further comprises dissolving the biodegradable polymer, and suspending the tropoelastin particles in the dissolved biodegradable polymer prior to the mixing step.

In some embodiments of any of the below- or above-mentioned embodiments, the method further comprises printing or casting the mixture.

In some embodiments of any of the below- or above-mentioned embodiments, the hybrid material is a yarn.

In some embodiments of any of the below- or above-mentioned embodiments, the method further comprises electrospinning the mixture, thereby forming an electrospun fibrous yarn.

In some embodiments of any of the below- or above-mentioned embodiments, the method further comprises collecting the electrospun fibrous yarn.

In some embodiments of any of the below- or above-mentioned embodiments, the method further comprises washing the hybrid material.

In some embodiments of any of the below- or above-mentioned embodiments, the mixture comprises a ratio of tropoelastin to biodegradable polymer of about 99:1, about 95:5, about 90:10, about 80:20, about 70:30, about 75:25, about 60:40, about 50:50, about 40:60, about 30:70, about 25:75, about 10:90 or about 0:100. In some embodiments of any of the below- or above-mentioned embodiments, the mixture comprises a ratio of tropoelastin to biodegradable polymer of about 99:1, 95:5, about 75:25, about 50:50, about 25:75 or about 0:100. In some embodiments of any of the below- or above-mentioned embodiments, the mixture comprises a ratio of tropoelastin to biodegradable polymer of about 50:50, about 25:75 or about 0:100. In some embodiments of any of the below- or above-mentioned embodiments, the mixture comprises a ratio of tropoelastin to biodegradable polymer of about 50:50. In some embodiments of any of the below- or above-mentioned embodiments, the mixture comprises a ratio of tropoelastin to biodegradable polymer of about 25:75. In some embodiments of any of the below- or above-mentioned embodiments, the mixture comprises a ratio of tropoelastin to biodegradable polymer of about 0:100.

In some embodiments of any of the below- or above-mentioned embodiments, the yarn or electrospun fibrous yarn comprises a length of about 1 cm, about 5 cm, about 15 cm, 15 cm, about 20 cm, about 25 cm, about 30 cm, about 35 cm, about 40 cm, about 45 cm, about 50 cm, about 75 cm, about 100 cm, about 125 cm, about 150 cm, about 175 cm, about 200 cm, about 225 cm, about 250 cm, about 275 cm, about 300 cm, about 325 cm, about 350 cm, about 375 cm, about 400 cm, about 425 cm, about 450 cm, about 475 cm, about 500 cm, about 525 cm, about 550 cm, about 575 cm, about 600 cm, about 625 cm, about 650 cm, about 675 cm, about 700 cm or any length in between a range defined by any two aforementioned values.

In some embodiments of any of the below- or above-mentioned embodiments, the method is performed at a relative humidity of between about 0% to about 5%, about 5% to about 10%, about 10% to about 15%, about 15% to about 20%, about 20% to about 25%, about 25% to about 30%, about 35% to about 40%, about 35% to about 40%, about 40% to about 45%, about 45% to about 50%, about 50% to about 55%, about 55% to about 60%, about or 60% to about 65%. In some embodiments of any of the below- or above-mentioned embodiments, the method is performed at a relative humidity of between about 35% to about 61%. In some embodiments of any of the below- or above-mentioned embodiments, the method is performed at a relative humidity of between about 42% to about 62%.

In some embodiments of any of the below- or above-mentioned embodiments, the ratio of tropoelastin to polycaprolactone (PCL) is about 75:25, about 50:50 or about 25:75. In some embodiments of any of the below- or above-mentioned embodiments, the ratio of tropoelastin to PCL is about 0:100.

In some embodiments of any of the below- or above-mentioned embodiments, the electrospinning is performed with an electrospinner comprising a funnel collector, wherein the funnel collector comprises a funnel collector speed of about 400 rpm, 425 rpm, 450 rpm, 475 rpm, 500 rpm, 525 rpm, 550 rpm, 575 rpm, 600 rpm, 625 rpm, 650 rpm, 675 rpm, 700 rpm, 725 rpm, 750 rpm, 775 rpm, 800 rpm, 825 rpm, 850 rpm, 875 rpm, 900 rpm, 925 rpm, 950 rpm, 975 rpm, 1000 rpm, or 1250 rpm or any speed in between a range defined by any two aforementioned values.

In some embodiments of any of the below- or above-mentioned embodiments, the electrospinner further comprises a rotating winder speed, wherein the rotating winder speed comprises a speed of about 2 rpm, 3 rpm, 4 rpm, 5 rpm, 6 rpm, 7 rpm, 8 rpm, 9 rpm, 10 rpm, 11 rpm, 12 rpm, or 13 rpm or any speed in between a range defined by any two aforementioned values.

In some embodiments of any of the below- or above-mentioned embodiments, the funnel collector speed and or rotating winder speed is adjusted depending on the relative humidity.

In some embodiments of any of the below- or above-mentioned embodiments, the mixing step is performed for at least about 4 hours. In some embodiments of any of the below- or above-mentioned embodiments, the mixing step is performed at about 4° C.

In a second aspect, a method of making a hybrid material is provided, the method comprises providing tropoelastin, providing a biodegradable polymer, melting the biodegradable polymer, thereby producing a melted biodegradable polymer, suspending the tropoelastin into the melted biodegradable polymer, producing a mixture and printing or casting the mixture; thereby producing a hybrid material.

In a third aspect, a method of making a hybrid material, the method comprises, providing tropoelastin, providing a biodegradable polymer, dissolving the tropoelastin, dissolving the biodegradable material, mixing the tropoelastin and biodegradable material thereby producing a mixture and printing or casting the mixture; thereby producing a hybrid material.

In a fourth aspect, a method of making a hybrid material is provided. The method comprises providing tropoelastin, providing a biodegradable polymer, dissolving the biodegradable polymer, suspending the tropoelastin into the biodegradable polymer, thereby producing a mixture and printing or casting the mixture; thereby producing a hybrid material.

In a fifth aspect, a method of making a hybrid material is provided. The method comprises providing tropoelastin, providing a biodegradable polymer, mixing the tropoelastin and biomaterial to produce a mixture, electrospinning the mixture and collecting the hybrid material in a form of an electrospun fibrous yarn.

In some embodiments of any of the below- or above-mentioned embodiments, the tropoelastin is provided as a monomer in solution.

In some embodiments of any of the below- or above-mentioned embodiments, the tropoelastin is provided as tropoelastin particles.

In a sixth aspect, a hybrid material is provided. The material comprises tropoelastin and a biodegradable polymer.

In some embodiments of any of the below- or above-mentioned embodiments, the hybrid material is a casted material. In some embodiments of any of the below- or above-mentioned embodiments, the hybrid material is a printed material. In some embodiments of any of the below- or above-mentioned embodiments, the hybrid material is an electrospun yarn.

In some embodiments of any of the below- or above-mentioned embodiments, the biodegradable polymer is PCL, poly(lactic acid), poly (lactic-co-glycolic acid, polyglycolic acid, poly(trimethylene carbonate, poly-4-hydroxybutyrate or a co-polymer of any one of the aforementioned polymers. In some embodiments of any of the below- or above-mentioned embodiments, the biodegradable polymer is PCL.

In some embodiments of any of the below- or above-mentioned embodiments, the PCL comprises a molecular weight of about 1,250 g/mol, 2,500 g/mol, 3,750 g/mol, 5,000 g/mol, 6,250 g/mol, 7,500 g/mol, 8,750 g/mol, 9,000 g/mol, 10,000 g/mol, 45,000 g/mol, 80,000 g/mol, 90,000 g/mol, or 100,000 g/mol. In some embodiments of any of the below- or above-mentioned embodiments, the PCL comprises a molecular weight of about 80,000 g/mol.

In some embodiments of any of the below- or above-mentioned embodiments, the hybrid material comprises a ratio of tropoelastin to biodegradable polymer of about 90:10, 80:20, 70:30, 75:25, 60:40, 50:50, 40:60, 30:70, 25:75, 10:90, or 0:100. In some embodiments of any of the below- or above-mentioned embodiments, the hybrid material comprises a ratio of tropoelastin to biodegradable polymer of about 75:25, 50:50, 25:75, or about 0:100. In some embodiments of any of the below- or above-mentioned embodiments, the hybrid material comprises a ratio of tropoelastin to biodegradable polymer of about 50:50, 25:75, or 0:100. In some embodiments of any of the below- or above-mentioned embodiments, the hybrid material comprises a ratio of tropoelastin to biodegradable polymer of about 50:50. In some embodiments of any of the below- or above-mentioned embodiments, the hybrid material comprises a ratio of tropoelastin to biodegradable polymer of about 25:75. In some embodiments of any of the below- or above-mentioned embodiments, the hybrid material comprises a ratio of tropoelastin to biodegradable polymer of about 0:100.

In some embodiments of any of the below- or above-mentioned embodiments, the hybrid material is biocompatible and biodegradable.

In some embodiments of any of the below- or above-mentioned embodiments, the hybrid material is non-toxic, and wherein breakdown products or by-products of the yarn do not interfere with tissue function.

In some embodiments of any of the below- or above-mentioned embodiments, the tropoelastin is monomeric. In some embodiments of any of the below- or above-mentioned embodiments, the tropoelastin is not crosslinked.

In some embodiments of any of the below- or above-mentioned embodiments, the hybrid material maintains structural integrity following exposure to aqueous solution.

In some embodiments of any of the below- or above-mentioned embodiments, the hybrid material maintains structural integrity at a temperature of at least about 37° C. In some embodiments of any of the below- or above-mentioned embodiments, the hybrid material maintains structural integrity at a temperature of about 37° C.

In some embodiments of any of the below- or above-mentioned embodiments, the hybrid material supports fibroblast growth. In some embodiments of any of the below- or above-mentioned embodiments, fibroblast growth is supported for at least about 7 days.

In some embodiments of any of the below- or above-mentioned embodiments, the hybrid material has a minimized foreign body response in tissue.

In some embodiments of any of the below- or above-mentioned embodiments, the hybrid material produces minimal inflammation in tissue.

In some embodiments of any of the below- or above-mentioned embodiments, the hybrid material is a yarn or an electrospun yarn, wherein the yarn or electrospun yarn comprises a fiber width of about 150 nm, 200 nm, 300 nm, 400 nm, 450 nm, 500 nm, 550 nm, 600 nm, 650 nm, 700 nm, 750 nm, 800 nm, 850 nm, 900 nm, 1000 nm, 1050 nm, 1100 nm, 1200 nm, 1400 nm, 1600 nm, 1800 nm, 2000 nm, 2500 nm, 3000 nm, 3500 nm, 4000 nm, 4500 nm, 5000 nm, 5500 nm, 6000 nm, 6500 nm, 7000 nm, 7500 nm, 8000 nm, 8500 nm, 9000 nm, 10,000 nm, or any fiber width in between a range defined by any two aforementioned values.

In some embodiments of any of the below- or above-mentioned embodiments, the hybrid material is a yarn or an electrospun yarn, wherein the yarn or electrospun yarn comprises a fiber twist angle of about 5°, about 10°, about 15°, about 20°, about 25°, about 30°, about 35°, about 40°, about 50°, about 55°, about 60°, about 65°, about 70°, about 75°, about 80°, about 85°, about 90°, about 95° or any angle in between a range defined by any two aforementioned values.

In some embodiments of any of the below- or above-mentioned embodiments, the hybrid material is a yarn or an electrospun yarn, wherein the yarn or electrospun yarn comprises a yarn width of about 50 μm, about 75 μm, about 100 μm, about 125 μm, about 150 μm, about 175 μm, about 200 μm, about 275 μm, 300 μm, about 325 μm, about 350 μm, about 375 μm, about 400 μm, about 425 μm, about 450 μm, about 475 μm, about 500 μm, about 525 μm, about 500 μm, about 525 μm, about 550 μm, about 575 μm, about 600 μm, about 625 μm, about 650 μm, about 675 μm, about 700 μm, about 725 μm, about 750 μm, about 775 μm, about 800 μm, about 825 μm, about 850 μm, about 875 μm, about 900 μm, about 925 μm, about 950 μm, about 975 μm or any yarn width in between a range defined by any two aforementioned values.

In some embodiments of any of the below- or above-mentioned embodiments, the biopolymer is absorbable.

In an eighth aspect, a tissue engineering scaffold for tissue repair is provided, the scaffold comprises a hybrid material, wherein the hybrid material comprises: tropoelastin and a biodegradable polymer.

In some embodiments of any of the below- or above-mentioned embodiments, the hybrid material is a printed material. In some embodiments of any of the below- or above-mentioned embodiments, the hybrid material is a casted material. In some embodiments of any of the below- or above-mentioned embodiments, the hybrid material is a yarn. In some embodiments of any of the below- or above-mentioned embodiments, the hybrid material is an electrospun yarn.

In some embodiments of any of the below- or above-mentioned embodiments, the biodegradable polymer comprises PCL.

In some embodiments of any of the below- or above-mentioned embodiments, the scaffold comprises a ratio of tropoelastin to biodegradable polymer of about 90:10, 80:20, 70:30, 75:25, 60:40, 50:50, 40:60, 30:70, 25:75, 10:90, or 0:100.

In some embodiments of any of the below- or above-mentioned embodiments, the scaffold is biocompatible and biodegradable.

In some embodiments of any of the below- or above-mentioned embodiments, the scaffold is non-toxic, and wherein breakdown products or by-products of the scaffold do not interfere with tissue function.

In some embodiments of any of the below- or above-mentioned embodiments, the scaffold supports in vitro fibroblast growth. In some embodiments of any of the below- or above-mentioned embodiments, the in vitro fibroblast growth is supported for at least about 7 days.

In some embodiments of any of the below- or above-mentioned embodiments, the scaffold provides a structure to allow cells to attach and infiltrate. In some embodiments of any of the below- or above-mentioned embodiments, the scaffold promotes cellular growth and cellular proliferation.

In some embodiments of any of the below- or above-mentioned embodiments, the scaffold provides structural support to cells and promotes repair of tissues by enabling tissues to attach to a surface of the scaffold and enables proliferation.

In some embodiments of any of the below- or above-mentioned embodiments, the scaffold has a low in vivo degradation rate, wherein the degradation is in excess of about two weeks or in excess of about four weeks.

In some embodiments of any of the below- or above-mentioned embodiments, the scaffold promotes elastogenesis and angiogenesis.

In some embodiments of any of the below- or above-mentioned embodiments, the scaffold does not lead to inflammation of the tissues and does not lead to foreign body response.

In some embodiments of any of the below- or above-mentioned embodiments, the scaffold comprises a hybrid yarn comprised of the tropoelastin and the biodegradable polymer.

In some embodiments of any of the below- or above-mentioned embodiments, the scaffold comprises an electrospun hybrid yarn comprised of the tropoelastin and the biodegradable polymer.

In some embodiments of any of the below- or above-mentioned embodiments, the scaffold comprises randomly arranged fibers of the hybrid yarn or electrospun hybrid yarn.

In some embodiments of any of the below- or above-mentioned embodiments, the scaffold comprises continuous yarns comprising the hybrid yarn or electrospun hybrid yarn, wherein the yarns comprise aligned fibers that are capable of withstanding mechanical stress.

In some embodiments of any of the below- or above-mentioned embodiments, the scaffold allows release of tropoelastin.

In a tenth aspect, a method of tissue repair is provided, the method comprises providing a tissue engineering scaffold, wherein the tissue engineering scaffold comprises a hybrid yarn, the yarn comprising: tropoelastin and a biodegradable polymer, and implanting the tissue engineering scaffold into tissue of an individual.

In some embodiments of any of the below- or above-mentioned embodiments, the biodegradable polymer comprises PCL, poly(lactic acid), poly (lactic-co-glycolic acid, polyglycolic acid, poly(trimethylene carbonate, poly-4-hydroxybutyrate or a co-polymer of any one of the aforementioned polymers. In some embodiments of any of the below- or above-mentioned embodiments, the biodegradable polymer comprises PCL.

In some embodiments of any of the below- or above-mentioned embodiments, the scaffold releases monomeric tropoelastin into the tissue of the individual.

In some embodiments of any of the below- or above-mentioned embodiments, the scaffold comprises a ratio of tropoelastin to biodegradable polymer of about 75:25, 50:50, 25:75, or 0:100. In some embodiments of any of the below- or above-mentioned embodiments, the scaffold comprises a ratio of tropoelastin to biodegradable polymer of about 50:50 or about 25:75.

In some embodiments of any of the below- or above-mentioned embodiments, the method promotes synthesis of new elastin in the tissue.

In some embodiments of any of the below- or above-mentioned embodiments, the method is performed for abdominal wall repair.

In some embodiments of any of the below- or above-mentioned embodiments, the method is performed for treating a hernia.

In some embodiments of any of the below- or above-mentioned embodiments, the tissue is vaginal tissue.

In an eleventh aspect, a scaffold for use in breast surgery is provided.

In some embodiments of any of the below- or above-mentioned embodiments, the breast surgery is a reconstruction surgery.

In some embodiments of any of the below- or above-mentioned embodiments, the breast surgery further comprises tissue expansion and/or a tissue expander.

In some embodiments of any of the below- or above-mentioned embodiments, the breast surgery comprises a vascular flap reconstruction.

In some embodiments of any of the below- or above-mentioned embodiments, the breast surgery comprises a breast augmentation with breast implants.

In some embodiments of any of the below- or above-mentioned embodiments, the scaffold supports one or a combination of a breast implant or breast tissue when used in reconstructive surgery.

In a twelfth aspect, a method of treating pelvic organ prolapse in an individual is provided, the method comprising: providing a tissue engineering scaffold, wherein the tissue engineering scaffold comprises a hybrid material, the hybrid material comprising: tropoelastin and PCL in a ratio of tropoelastin to PCL of about 25:75, placing the scaffold into vaginal tissue of the individual.

In some embodiments of any of the below- or above-mentioned embodiments, the hybrid material comprises an electrospun hybrid yarn.

In some embodiments of any of the below- or above-mentioned embodiments, the method promotes deposition of collagen into the tissue of the individual.

In some embodiments of any of the below- or above-mentioned embodiments, the method promotes deposition of collagen around the scaffold.

In some embodiments of any of the below- or above-mentioned embodiments, the method promotes an anti-inflammatory effect in the tissue surrounding the scaffold.

In some embodiments of any of the below- or above-mentioned embodiments, the method promotes localization of macrophages at an interface between the scaffold and the tissue.

In some embodiments of any of the below- or above-mentioned embodiments, the method promotes tissue regeneration.

In some embodiments of any of the below- or above-mentioned embodiments, the pelvic organ prolapse is caused by a dropped bladder (cystocele).

In some embodiments of any of the below- or above-mentioned embodiments, the pelvic organ prolapse is caused by rectocele.

In some embodiments of any of the below- or above-mentioned embodiments, the pelvic organ prolapse is caused by a dropped uterus (uterine prolapse).

In some embodiments of any of the below- or above-mentioned embodiments, the tissue engineering scaffold has a Young's modulus similar to the Young's modulus of the vaginal tissue.

In some embodiments of any of the below- or above-mentioned embodiments, the tissue engineering scaffold has a Young's modulus of about 30 MPa, about 31 MPa, about 32 MPa, about 33 MPa, about 34 MPa, about 35 MPa, about 36 MPa, about 37 MPa, about 38 MPa, about 39 MPa, or about 40 MPa.

### BRIEF DESCRIPTION OF THE DRAWINGS

FIGS. 1A and 1B. Images of poorly formed tropoelastin:PCL electrospun yarns. (1A) 25:75 yarn produced using initial electrospinning parameters. (1B) 0:100 yarn produced in relative humidity levels below working range.

FIGS. 2A-2E. Images of various blends of tropoelastin:PCL electrospun yarns produced using optimized electrospinning parameters. (2A) 621 cm long 50:50 electrospun yarn wound around rotating winder. (2B) to (2E) electrospun yarns wound around storage tubes. (2B) 75:25 electrospun yarn, (2C) 50:50 electrospun yarn, (2D) 25:75 electrospun yarn, (2E) 0:100 electrospun yarn.

FIGS. 3A-3C: Measurements from SEM micrographs of 75:25, 50:50, 25:75 and 0:100 tropoelastin:PCL electrospun yarns. (3A) fiber width, (3B) fiber angle, (3C) yarn width. Data show the mean±standard deviation. For each group n=3.

FIGS. 4A-4P: SEM micrographs of tropoelastin:PCL electrospun yarns before and after water treatments. (4A) 75:25 yarns untreated, (4B) 75:25 yarns immersed in MQW for 24 hours at 37° C., (4C) 20° C., or (4D) 4° C. (4E) 50:50 yarns untreated, (4F) 75:25 yarns immersed in MQW for 24 hours at 37° C., (4G) 20° C., or (4H) 4° C. (4I) 25:75 yarns untreated, (4J) 75:25 yarns immersed in MQW for 24 hours at 37° C., (4K) 20° C., or (4L) 4° C. (4M) 0:100 yarns untreated, (4N) 75:25 yarns immersed in MQW for 24 hours at 37° C., (40) 20° C., or (4P) 4° C. Representative images, n=1.

FIGS. 5A-5F: Images of electrospun 50:50 tropoelastin:PCL yarn (5A) woven into mesh (5B). The yarn and mesh were hydrated in PBS at room temperature and their mechanical properties were determined including, Young's modulus (5C), ultimate tensile strength (5D), and elongation (5E). Cyclic tensile testing was performed on the mesh with stress (MPa) plotted again strain CA) (n=3 or 4 for each condition) (5F).

FIGS. 6A-6C: (6A) Comparison of FTIR-ATR offset spectra of tropoelastin:PCL electrospun yarns and pure tropoelastin. Representative FTIR-ATR spectra over the wavenumber range 1950-1350 cm-1. (6B) FTIR-ATR spectral peak height of Amide I band of tropoelastin:PCL electrospun yarns. (6C) Spectral peak height of carbonyl group band of tropoelastin:PCL electrospun yarns. Data show the mean±standard deviation, for each group n=3.

FIGS. 7A-7D: SDS-PAGE analysis of protein released from tropoelastin:PCL electrospun yarns after sterilization in absolute ethanol and then incubation in PBS at 37° C., 20° C. or 4° C. for 1 or 7 days. (7A) 75:25 yarns, (7B) 50:50 yarns, (7C) 25:75 yarns, (7D) 0:100 yarns. Lane 1: tropoelastin monomer (0.25 mg/mL), lane 8: Mark12™ protein standard.

FIGS. 8A-8B: (8A) Tropoelastin remaining in tropoelastin:PCL electrospun yarns after incubation in PBS at 37° C., 20° C. or 4° C. for 7 days. (8B) Tropoelastin remaining in tropoelastin:PCL electrospun yarns after incubation in PBS at 37° C. for 7 days. Data expressed as tropoelastin remaining (mg) in 1 mg section of yarn. Data show the mean±standard deviation. For each group n=3.

FIG. 9: Confocal images of human dermal fibroblasts cultured on 75:25, 50:50, 25:75 or 0:100 tropoelastin:PCL electrospun yarns after 7 days incubation in cell culture media at 37° C. Cells were stained with ActinRed™ (red) to view F-Actin and TO-PRO™ 3 iodide (cyan) to image nuclei. (Merged images). Representative images, n=1.

FIGS. 10A-10R: Histology of tropoelastin:PCL scaffold after 4 weeks implantation in the ovine vagina. (10A, 10C, 10D) H&E (a) showing panoramic view of 25:75 tropoelastin:PCL mesh mainly between the lamina propria and muscularis (black arrows) and in the muscularis (three arrows on bottom right corner). (10B) incision control and at (10E, 10F) higher power). Collagen staining by (10G-10J) Gomori (blue) and (10K-10N) Sirius Red (red) showing (10G, 10H, 10K, 109L) collagen around mesh filaments (arrows) and in ECM and in the (10I, 10J, 10M, 10N) incision control. (10O-10R) Verhoff van Gieson (VVG) staining showing (10O, 10P) a few black elastin fibres in the tissue and (10O, 10P) around the tropoelastin of the mesh filament surface. LP, lamina propria. Representative images n=1 each of scaffold implanted and incision control ewes. Scale bars; (10A-10B) 2 mm, (10C-10R) 200 μm

FIGS. 11A-11F: Immunofluorescence images showing deposition of collagen III in explanted ovine vaginal tissue after 30 days (11A) near the incision site and (11B) around filaments of the tropoelastin:PCL scaffold. (11C) Isotype control. SEM micrographs of explanted ovine vaginal tissue with (11D) tropoelastin: PCL scaffold showing (11E) integrity of yarn structure and (11F) integration (white dotted box) of scaffold (#) with host tissue (*) after 30 days. Dotted line indicates epithelial lamina propria border. e, epithelium; t, tropoelastin:PCL.

FIGS. 12A-12F: Minimal foreign body response to an implanted tropoelastin:PCL scaffold in an ovine vaginal surgery model of POP. Immunohistochemistry for CD45+ leukocytes (brown) in the (12A) epithelium and lamina propria of a tropoelastin:PCL explant and (12C) incision control and (12B, 12D) CD206+M2 macrophages (brown). Colocalization of (12E) CD45+ leukocytes (green) and CD206+M2 macrophages (red, merge, yellow) at the tropoelastin:PCL filament tissue interface. In tissue more distant to the scaffold filaments, CD45+ leukocytes (green in merge panel) were either M1 inflammatory or M0 uncommitted macrophages. (f) CD45+ leukocytes (green) colocalise with CD206+M2 macrophages (red). Representative images of n=1 scaffold implanted and n=1 incision control ewe.

FIG. 13: Examples of pre-implantation woven scaffolds made from tropoelastin:PCL electrospun yarns.

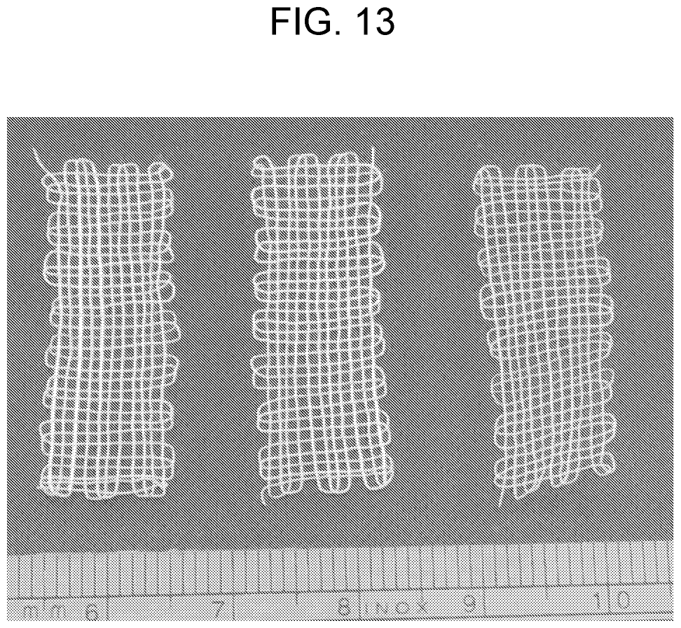

### DETAILED DESCRIPTION

Pelvic organ prolapse (POP) is a debilitating condition that may affect 25% of all women (Jelovsek et al. The Lancet (2007) 369 (9566), 1027; incorporated by reference in its entirety herein). POP occurs when the pelvic support structures; suspensory ligaments, vaginal wall and pelvic floor muscles are damaged. Without being limiting, damage may occur from vaginal birth and weaken over time, causing the downward descent of pelvic organs (Dwyer et al. Obstetrics, Gynaecology & Reproductive Medicine (2018) 28 (1), 15; incorporated by reference in their entirety herein). Symptoms may include, but are not limited to bladder, bowel and sexual dysfunction, feeling of a bulge in the vagina and less commonly urinary and fecal incontinence, for example. Risk factors for POP may include childbirth, obesity and increased age, for example. Non-degradable synthetic meshes have been used for decades for abdominal hernia repair and more recently for the surgical repair of vaginal tissue in women with POP, however their use is now severely restricted due to company withdrawal of vaginal mesh and regulatory authority bans on their use in the USA, UK, Australia and New Zealand. Reported complications leading to these bans were mesh erosion into pelvic organs, mesh exposure, infections and pain requiring further surgeries for their removal (Ganj et al. Int Urogynecol J Pelvic Floor Dysfunct (2009) 20 (8), 919 and Silva et al. Current Opinion in Obstetrics and Gynecology (2005) 17, 519; incorporated by reference; incorporated by reference in their entirety herein). About 20% of women requiring POP reconstructive surgery are now faced with limited treatment options as native tissue surgery fails in about ˜30% of cases.

Tissue engineering scaffolds promote tissue repair by providing a surface for cells to attach to and proliferate (O'Brien et al. Materials Today (2011) 14 (3), 88 and Freed et al. Nature (1994) 12, 689; incorporated by reference in their entirety herein). Scaffolds must meet a number of criteria to be successful for tissue engineering applications. Surface structure of a scaffold affects the ability of cells to adhere and proliferate (Rnjak-Kovacina et al. Biomaterials (2011) 32 (28), 6729; incorporated by reference in its entirety), whereby a fibrous and porous structure may enable cells to attach and infiltrate throughout the scaffold. Furthermore, an ideal scaffold will need to be degradable to allow for growth of new tissue and also to avoid the need for surgical removal (Ulery et al. J Polym Sci B Polym Phys (2011) 49 (12), 832; incorporated by reference in its entirety). The materials used may be non-toxic, and any by-products produced during the breakdown should not interfere with or harm the surrounding tissue at implant site (O'Brien et al. Materials Today (2011 14(3), 88 and Liu et al. International Journal of Nanomedicine (2006) 1 (4), 541; incorporated by reference in its entirety herein). In addition, the scaffold may be biocompatible to allow cells to attach and populate the scaffold (O'Brien et al. Materials Today (2011) 14(3), 88; incorporated by reference in its entirety herein). The scaffold may possess mechanical properties to match the mechanical requirements of the native tissue (Hutmacher et al. Biomaterials (2000) 21, 2529 and Wu et al. Acta Biomater (2017) 62, 102; incorporated by reference in their entirety herein). Various factors influence mechanical properties of a scaffold, such as, for example, the physical properties of the material components themselves, the proportions of each component within the scaffold, as well as material degradation. Each of these factors must be taken into consideration when selecting an ideal scaffold for an intended application. The ability to create an elastic and biocompatible mesh with strength to support organs in the body is a need that remains unmet.

Electrospinning is a technique used to fabricate fibrous scaffolds (Baumgarten et al. Journal of Colloid and Interface Science (1971) 36 (1), 71; incorporated by reference in its entirety herein). These scaffolds consist of randomly arranged fibers (Wu et al. Acta Biomater (2017) 62, 102; incorporated by reference in its entirety herein), and whilst suitable for applications such as dermal wound repair, scaffolds like these provide limited mechanical strength. As a result, they are not suitable for the repair of load-bearing tissues in the body. A continuous yarn may be fabricated using a modified electrospinning set up as described previously (Ali et al Journal of the Textile Institute (2012) 103 (1), 80 incorporated by reference in its entirety herein). These continuous yarns consist of aligned fibers that form a twist which increases tensile strength and flexibility of the yarn. These continuous yarns may be capable of being woven into more complex structures and possess the ability to withstand the mechanical stress necessary to act as a scaffold for load-bearing tissues in the body (Ali et al Journal of the Textile Institute (2012) 103 (1), 80 and Moutos et al. Biorheology (2008) 45 (3-4), 501: incorporated by reference their its entirety herein). This may be an important consideration for vaginal repair.

Elastin is one of the components that make up the extracellular matrix (ECM) and is found throughout the body such as in skin and blood vessels, where it provides elasticity to these tissues so they can withstand continuous strain (Rodgers et al. Pathol Biol (Paris) (2005) 53 (7), 390 and Shen et al. Scaffold and Biomechanical Transductive Approaches to Elastic Tissue Engineering. In Elastic Fiber Matrices, Anand Ramamurthi, C. K., (ed.) CRC Press, Taylor & Francis Group (2016); incorporated by reference in their entirety herein). Elastin is cell interactive and influences cellular attachment (Wise et al. Acta Biomater (2014) 10 (4), 1532 and Bax et al. J Biol Chem (2009) 284 (42), 28616; incorporated by reference in their entirety herein), proliferation (Rodgers et al. 2005) and differentiation (Jin et al. Regenerative Engineering and Translational Medicine (2016) 2 (2), 85; incorporated by reference in its entirety herein. Tropoelastin, the soluble monomeric subunit of elastin, has similar biological and physical properties to elastin that are preserved after electrospinning (Yeo et al. Advanced Healthcare Materials (2015) 4 (16), 2530; incorporated by reference in its entirety herein). Tropoelastin electrospun scaffolds have been shown to support cell growth and promote proliferation and are also well tolerated in vivo (Li et al. Biomaterials (2005) 26 (30), 5999; Rnjak-Kovacina et al. Biomaterials (2011) 32 (28), 6729; Liu et al. Cytokine (2014) 70 (1), 55; incorporated by reference in their entirety herein).

Polycaprolactone (PCL) is a synthetic, non-toxic degradable polymer that has been approved for use in certain biomedical applications by the US Food and Drug Administration (Ulery et al. J Polym Sci B Polym Phys (2011) 49 (12), 832, Diaz et al. Journal of Nanomaterials (2014) 2014, 1; Ghosal et al. AAPS PharmSciTech (2017) 18 (1), 72; incorporated by reference in its entirety herein). PCL has been used in electrospinning to fabricate scaffolds that have a low in vivo degradation rate and have been successfully used in dermal tissues and tendon repair (Bolgen et al. Journal of Biomaterials Science, Polymer Edition (2005) 16 (12), 1537; Ghosal et al. AAPS PharmSciTech (2017) 18 (1), 72; Wu et al. Acta Biomater (2017) 62, 102; incorporated by reference in their entirety herein). However, PCL is a synthetic polymer, and thus, it is hydrophobic and lacks cell adhesion sites (Bolgen et al. Journal of Biomaterials Science, Polymer Edition (2005) 16 (12), 1537; Zhang et al. Biomacromolecules (2005) 6, 2583; incorporated by reference in its entirety herein). PCL is may be blended with natural polymers to improve biocompatibility (Ghosal et al. AAPS PharmSciTech (2017) 18 (1), 72; Zhang et al. Biomacromolecules (2005) 6, 2583; incorporated by reference in its entirety herein).

As described in the embodiments herein, the biological and physical properties of tropoelastin are combined with the favorable physical properties of PCL to produce hybrid yarns in multi-meter lengths that may be degradable and capable of supporting cellular growth. It is also shown, for the first time, the potential of these hybrid yarns as a vaginal scaffold for tissue engineering applications in an ovine model of POP.

Unless defined otherwise, all technical and scientific terms used herein have the same meaning as commonly understood by one of ordinary skill in the art to which the disclosure pertains.

The terms “a,” “an,” “the” and similar referents used in the context of describing the disclosure (especially in the context of the following Claims) are to be construed to cover both the singular and the plural, unless otherwise indicated herein or clearly contradicted by context. “About” as used herein when referring to a measurable value is meant to encompass variations of +20% or +10%, more preferably +5%, even more preferably +1%, and still more preferably +0.1% from the specified value.

As used herein, except where the context requires otherwise, the term ‘comprise’ and variations of the term, such as “comprising,” “comprises” and “comprised,” are not intended to exclude further additives, components, integers or steps.

The term “tropoelastin” refers to a protein from which elastin is formed. Tropoelastin may be monomeric. Tropoelastin is generally not cross-linked, covalently or otherwise. Tropoelastin may reversibly coacervate. Thus, tropoelastin is distinguished from elastin because elastin consists of covalently cross linked tropoelastin which cannot reversibly coacervate. The tropoelastin may be human tropoelastin. Tropoelastin may be synthetic, for example it may be derived from recombinant expression or other synthesis, or it may be obtained from a natural source such as porcine aorta. As generally known in the art, tropoelastin may exist in the form of a variety of fragments. In some embodiments of each or any of the above- or below-mentioned embodiments, the composition provided in the methods herein comprises monomeric tropoelastin. In some embodiments, the tropoelastin is particulate. In further embodiments the tropoelastin is non-particulate. In still further embodiments, the tropoelastin is a powder. In some embodiments of each or any of the above- or below-mentioned embodiments, the tropoelastin comprises the sequence set forth in any one of SEQ ID NOs: 1-15.

In some embodiments of each or any of the above- or below-mentioned embodiments, the methods of the disclosure utilize the SHELδ26A tropoelastin analogue (WO 1999/03886) for the various applications described herein including for the compositions that are used in the described methods. The amino acid sequence of SHELδ26A is:

(SEQ ID NO: 1)
GGVPGAIPGGVPGGVFYPGAGLGALGGGALGPGGKP
LKPVPGGLAGAGLGAGLGAFPAVTFPGALVPGGVAD
AAAAYKAAKAGAGLGGVPGVGGLGVSAGAVVPQPGA
GVKPGKVPGVGLPGVYPGGVLPGARFPGVGVLPGVP
TGAGVKPKAPGVGGAFAGIPGVGPFGGPQPGVPLGY
PIKAPKLPGGYGLPYTTGKLPYGYGPGGVAGAAGKA
GYPTGTGVGPQAAAAAAAKAAAKFGAGAAGVLPGVG
GAGVPGVPGAIPGIGGIAGVGTPAAAAAAAAAAKAA
KYGAAAGLVPGGPGFGPGVVGVPGAGVPGVGVPGAG
IPVVPGAGIPGAAVPGVVSPEAAAKAAAKAAKYGAR
PGVGVGGIPTYGVGAGGFPGFGVGVGGIPGVAGVPS
VGGVPGVGGVPGVGISPEAQAAAAAKAAKYGVGTPA
AAAAKAAAKAAQFGLVPGVGVAPGVGVAPGVGVAPG
VGLAPGVGVAPGVGVAPGVGVAPGIGPGGVAAAAKS
AAKVAAKAQLRAAAGLGAGIPGLGVGVGVPGLGVGA
GVPGLGVGAGVPGFGAVPGALAAAKAAKYGAAVPGV
LGGLGALGGVGIPGGVVGAGPAAAAAAAKAAAKAAQ
FGLVGAAGLGGLGVGGLGVPGVGGLGGIPPAAAAKA
AKYGAAGLGGVLGGAGQFPLGGVAARPGFGLSPIFP
GGACLGKACGRKRK.

In some embodiments of each or any of the above- or below-mentioned embodiments, the tropoelastin isoform is the SHEL isoform (WO 1994/14958; included by reference in its entirety herein):

(SEQ ID NO: 2)
SMGGVPGAIPGGVPGGVFYPGAGLGALGGGALGPGG
KPLKPVPGGLAGAGLGAGLGAFPAVTFPGALVPGGV
ADAAAAYKAAKAGAGLGGVPGVGGLGVSAGAVVPQP
GAGVKPGKVPGVGLPGVYPGGVLPGARFPGVGVLPG
VPTGAGVKPKAPGVGGAFAGIPGVGPFGGPQPGVPL
GYPIKAPKLPGGYGLPYTTGKLPYGYGPGGVAGAAG
KAGYPTGTGVGPQAAAAAAAKAAAKFGAGAAGVLPG
VGGAGVPGVPGAIPGIGGIAGVGTPAAAAAAAAAAK
AAKYGAAAGLVPGGPGFGPGVVGVPGAGVPGVGVPG
AGIPVVPGAGIPGAAVPGVVSPEAAAKAAAKAAKYG
ARPGVGVGGIPTYGVGAGGFPGFGVGVGGIPGVAGV
PSVGGVPGVGGVPGVGISPEAQAAAAAKAAKYGVGT
PAAAAAKAAAKAAQFGLVPGVGVAPGVGVAPGVGVA
PGVGLAPGVGVAPGVGVAPGVGVAPGIGPGGVAAAA
KSAAKVAAKAQLRAAAGLGAGIPGLGVGVGVPGLGV
GAGVPGLGVGAGVPGFGAGADEGVRRSLSPELREGD
PSSSQHLPSTPSSPRVPGALAAAKAAKYGAAVPGVL
GGLGALGGVGIPGGVVGAGPAAAAAAAKAAAKAAQF
GLVGAAGLGGLGVGGLGVPGVGGLGGIPPAAAAKAA
KYGAAGLGGVLGGAGQFPLGGVAARPGFGLSPIFPG
GACLGKACGRKRK

or a protease resistant derivative of the SHEL or SHELδ26A isoforms (WO 2000/04043; included by reference in its entirety herein). As described in WO 2000/04043, the protein sequences of tropoelastin described may have a mutated sequence that leads to a reduced or eliminated susceptibility to digestion by proteolysis. Without being limiting, the tropoelastin amino acid sequence has a reduced or eliminated susceptibility to serine proteases, thrombin, kallikrein, metalloproteases, gelatinase A, gelatinase B, serum proteins, trypsin or elastase, for example. In some embodiments of each or any of the above- or below-mentioned embodiments, the tropoelastin comprises a sequence set forth in SEQ ID NO: 3 (SHELδ26A isoform):

(SEQ ID NO: 3)
GGVPGAIPGGVPGGVFYPGAGLGALGGGALGPGGKP
LKPVPGGLAGAGLGAGLGAFPAVTFPGALVPGGVAD
AAAAYKAAKAGAGLGGVPGVGGLGVSAGAVVPQPGA
GVKPGKVPGVGLPGVYPGGVLPGARFPGVGVLPGVP
TGAGVKPKAPGVGGAFAGIPGVGPFGGPQPGVPLGY
PIKAPKLPGGYGLPYTTGKLPYGYGPGGVAGAAGKA
GYPTGTGVGPQAAAAAAAKAAAKFGAGAAGVLPGVG
GAGVPGVPGAIPGIGGIAGVGTPAAAAAAAAAAKAA
KYGAAAGLVPGGPGFGPGVVGVPGAGVPGVGVPGAG
IPVVPGAGIPGAAVPGVVSPEAAAKAAAKAAKYGAR
PGVGVGGIPTYGVGAGGFPGFGVGVGGIPGVAGVPS
VGGVPGVGGVPGVGISPEAQAAAAAKAAKYGVGTPA
AAAAKAAAKAAQFGLVPGVGVAPGVGVAPGVGVAPG
VGLAPGVGVAPGVGVAPGVGVAPGIGPGGVAAAAKS
AAKVAAKAQLRAAAGLGAGIPGLGVGVGVPGLGVGA
GVPGLGVGAGVPGFGAVPGALAAAKAAKYGAAVPGV
LGGLGALGGVGIPGGVVGAGPAAAAAAAVLGGAGQF
PLGGVAARPGFGLSPIFPGGACLGKACGRKRK.

In some embodiments, the tropoelastin comprises a sequence set forth in SEQ ID NO: 4 (SHELδ mod isoform):

(SEQ ID NO: 4)
GGVPGAVPGGVPGGVFYPGAGFGAVPGGVADAAAAY
KAAKAGAGLGGVPGVGGLGVSAGAVVPQPGAGVKPG
KVPGVGLPGVYPGFGAVPGARFPGVGVLPGVPTGAG
VKPKAPGVGGAFAGIPGVGPFGGPQPGVPLGYPIKA
PKLPGGYGLPYTTGKLPYGYGPGGVAGAAGKAGYPT
GTGVGPQAAAAAAAKAAAKFGAGAAGFGAVPGVGGA
GVPGVPGAIPGIGGIAGVGTPAAAAAAAAAAKAAKY
GAAAGLVPGGPGFGPGVVGVPGFGAVPGVGVPGAGI
PVVPGAGIPGAAGFGAVSPEAAAKAAAKAAKYGARP
GVGVGGIPTYGVGAGGFPGFGVGVGGIPGVAGVPSV
GGVPGVGGVPGVGISPEAQAAAAAKAAKYGVGTPAA
AAAKAAAKAAQFGLVPGVGVAPGVGVAPGVGVAPGV
GLAPGVGVAPGVGVAPGVGVAPGIGPGGVAAAAKSA
AKVAAKAQLRAAAGLGAGIPGLGVGVGVPGLGVGAG
VPGLGVGAGVPGFGAVPGALAAAKAAKYGAVPGVLG
GLGALGGVGIPGGVVGAGPAAAAAAAKAAAKAAQFG
LVGAAGLGGLGVGGLGVPGVGGLGGIPPAAAAKAAK
YGAAGLGGVLGGAGQFPLGGVAARPGFGLSPIFPGG
ACLGKACGRKRK.

In some embodiments of each or any of the above- or below-mentioned embodiments, the tropoelastin may have at least 90% sequence identity with the amino acid sequence of a human tropoelastin isoform across at least 50 consecutive amino acids. It may, for example, have the sequence of a human tropoelastin isoform.

Tropoelastin analogues generally have a sequence that is homologous to a human tropoelastin sequence. Percentage identity between a pair of sequences may be calculated by the algorithm implemented in the BESTFIT computer program. Another algorithm that calculates sequence divergence has been adapted for rapid database searching and implemented in the BLAST computer program. In comparison to the human sequence, the tropoelastin polypeptide sequence may be about 60% identical at the amino acid level, 70% or more identical at the amino acid level, 80% or more identical at the amino acid level, 90% or more identical at the amino acid level, 95% or more identical at the amino acid level, 97% or more identical at the amino acid level, or greater than 99% identical at the amino acid level.

Recombinant forms of tropoelastin can be produced as shown in WO 1999/03886. These sequences are:

(SEQ ID NO: 5)
SMGGVPGAIPGGVPGGVFYPGAGLGALGGGALGPGG
KPLKPVPGGLAGAGLGAGLGAFPAVTFPGALVPGGV
ADAAAAYKAAKAGAGLGGVPGVGGLGVSAGAVVPQP
GAGVKPGKVPGVGLPGVYPGGVLPGARFPGVGVLPG
VPTGAGVKPKAPGVGGAFAGIPGVGPFGGPQPGVPL
GYPIKAPKLPGGYGLPYTTGKLPYGYGPGGVAGAAG
KAGYPTGTGVGPQAAAAAAAKAAAKFGAGAAGVLPG
VGGAGVPGVPGAIPGIGGIAGVGTPAAAAAAAAAAK
AAKYGAAAGLVPGGPGFGPGVVGVPGAGVPGVGVPG
AGIPVVPGAGIPGAAVPGVVSPEAAAKAAAKAAKYG
ARPGVGVGGIPTYGVGAGGFPGFGVGVGGIPGVAGV
PSVGGVPGVGGVPGVGISPEAQAAAAAKAAKYGVGT
PAAAAAKAAAKAAQFGLVPGVGVAPGVGVAPGVGVA
PGVGLAPGVGVAPGVGVAPGVGVAPGIGPGGVAAAA
KSAAKVAAKAQLRAAAGLGAGIPGLGVGVGVPGLGV
GAGVPGLGVGAGVPGFGAGADEGVRRSLSPELREGD
PSSSQHLPSTPSSPRVPGALAAAKAAKYGAAVPGVL
GGLGALGVGIPGGVVGAGPAAAAAAAKAAAKAAQFG
LVGAAGLGGLGVGGLGVPGVGGLGGIPPAAAAKAAK
YGAAGLGGVLGGAGQFPLGGVAARPGFGLSPIFPGG
ACLGKACGRKRK;
(SEQ ID NO: 6)
GGVPGAIPGGVPGGVFYPGAGLGALGGGALGPGGKP
LKPVPGGLAGAGLGAGLGAFPAVTFPGALVPGGVAD
AAAAYKAAKAGAGLGGVPGVGGLGVSAGAVVPQPGA
GVKPGKVPGVGLPGVYPGGVLPGARFPGVGVLPGVP
TGAGVKPKAPGVGGAFAGIPGVGPFGGPQPGVPLGY
PIKAPKLPGGYGLPYTTGKLPYGYGPGGVAGAAGKA
GYPTGTGVGPQAAAAAAAKAAAKFGAGAAGVLPGVG
GAGVPGVPGAIPGIGGIAGVGTPAAAAAAAAAAKAA
KYGAAAGLVPGGPGFGPGVVGVPGAGVPGVGVPGAG
IPVVPGAGIPGAAVPGVVSPEAAAKAAAKAAKYGAR
PGVGVGGIPTYGVGAGGFPGFGVGVGGIPGVAGVPS
VGGVPGVGGVPGVGISPEAQAAAAAKAAKYGVGTPA
AAAAKAAAKAAQFGLVPGVGVAPGVGVAPGVGVAPG
VGLAPGVGVAPGVGVAPGVGVAPGIGPGGVAAAAKS
AAKVAAKAQLRAAAGLGAGIPGLGVGVGVPGLGVGA
GVPGLGVGAGVPGFGAVPGALAAAKAAKYGAAVPGV
LGGLGALGGVGIPGGVVGAGPAAAAAAAKAAAKAAQ
FGLVGAAGLGGLGVGGLGVPGVGGLGGIPPAAAAKA
AKYGAAGLGGVLGGAGQFPLGGVAARPGFGLSPIFP
GGACLGKACGRKRK;
(SEQ ID NO: 7)
MGGVPGAVPGGVPGGVFYPGAGFGAVPGGVADAAAA
YKAAKAGAGLGGVPGVGGLGVSAGAVVPQPGAGVKP
GKVPGVGLPGVYPGFGAVPGARFPGVGVLPGVPTGA
GVKPKAPGVGGAFAGIPGVGPFGGPQPGVPLGYPIK
APKLPGGYGLPYTTGKLPYGYGPGGVAAAGKAGYPT
GTGVGPQAAAAAAAKAAAKFGAGAAGFGAVPGVGGA
GVPGVPGAIPGIGGIAGVGTPAAAAAAAAAAKAAKY
GAAAGLVPGGPGFGPGVVGVPGFGAVPGVGVPGAGI
PVVPGAGIPGAAGFGAVSPEAAAKAAAKAAKYGARP
GVGVGGIPTYGVGAGFFPGFGVGVGGIPGVAGVPSV
GGVPGVGGVPGVGISPEAQAAAAAKAAKYGVGTPAA
AAAKAAAKAAQFGLVPGVGVAPGVGVAPGVGVAPGV
GLAPGVGVAPGVGVAPGVGVAPGIGPGGVAAAAKSA
AKVAAKAQLRAAAGLGAGIPGLGVGVGVPGLGVGAG
VPGLGVGAGVPGFGAVPGALAAAKAAKYGAVPGVLG
GLGALGGVGIPGGVVGAGPAAAAAAAKAAAKAAQFG
LVGAAGLGGLGVGGLGVPGVGGLGGIPPAAAAKAAK
YGAAGLGGVLGGAGQFPLGGVAARPGFGLSPIFPGG
ACLGKACGRKRK;
(SEQ ID NO: 8)
SAMGGVPGALAAAKAAKYGAAVPGVLGGLGALGGVG
IPGGVVGAGPAAAAAAAKAAAKAAQFGLVGAAGLGG
LGVGGLGVPGVGGLGGIPPAAAAKAAKYGAAGLGGV
LGGAGQFPLGGVAARPGFGLSPIFPGGACLGKACGR
KRK;
(SEQ ID NO: 9)
SAMGALVGLGVPGLGVGAGVPGFGAGADEGVRRSLS
PELREGDPSSSQHLPSTPSSPRVPGALAAAKAAKYG
AAVPGVLGGLGALGGVGIPGGVVGAGPAAAAAAAKA
AAKAAQFGLVGAAGLGGLGVGGLGVPGVGGLGGIPP
AAAAKAAKYGAAGLGGVLGGAGQFPLGGVAARPGFG
LSPIFPGGACLGKACGRKRK;
(SEQ ID NO: 10)
GIPPAAAAKAAKYGAAGLGGVLGGAGQFPLGGVAAR
PGFGLSPIFPGGACLGKACGRKRK;
(SEQ ID NO: 11)
GAAGLGGVLGGAGQFPLGGVAARPGFGLSPIFPG
GACLGKACGRKRK;
(SEQ ID NO: 12)
GADEGVRRSLSPELREGDPSSSQHLPSTPSSPRV;
(SEQ ID NO: 13)
GADEGVRRSLSPELREGDPSSSQHLPSTPSSPRF;
(SEQ ID NO: 14)
AAAGLGAGIPGLGVGVGVPGLGVGAGVPGLGVGAGV
PGFGAGADEGVRRSLSPELREGDPSSSQHLPSTPSS
PRVPGALAAAKAAKYGAAVPGVLGGLGALGGVGIPG
GVVGAGPAAAAAAAKAAAKAAQFGLVGAAGLGGLGV
GGLGVPGVGGLGGIPPAAAAKAAKYGAAGLGGVLGG
AGQFPLGGVAARPGFGLSPIFPGGACLGKACGRKRK;
and
(SEQ ID NO: 15)
AAAGLGAGIPGLGVGVGVPGLGVGAGVPGLGVGAGV
PGFGAVPGALAAAKAAKYGAAVPGVLGGLGALGGVG
IPGGVVGAGPAAAAAAAKAAAKAAQFGLVGAAGLGG
LGVGGLGVPGVGGLGGIPPAAAAKAAKYGAAGLGGV
LGGAGQFPLGGVAARPGFGLSPIFPGGACLGKACGR
KRK.

“Biodegradable polymer” is a polymer that breaks down via natural processes that may result in natural by products. Without being limiting, biodegradable polymers may include PCL, poly(lactic acid), poly (lactic-co-glycolic acid, polyglycolic acid, poly(trimethylene carbonate, or poly-4-hydroxybutyrate, for example.

“Printing” or “3D printing” is a process wherein material is joined or solidified under computer control to create a three-dimensional object, with material being added together (such as liquid molecules or powder grains being fused together), typically layer by layer, for example.

“Casting” refers to a process in which a liquid material is usually poured into a mold, which contains a hollow cavity of the desired shape, and then allowed to solidify. The solidified part is also known as a casting, which is ejected or broken out of the mold to complete the process.

“Electrospinning” is a method to produce ultrafine (in nanometers) fibres by charging and ejecting a polymer melt or solution through a spinneret under a high-voltage electric field and to solidify or coagulate it to form a filament or an electrospun yarn.

In some embodiments of any of the below- or above-mentioned embodiments, a hybrid yarn is provided, wherein the yarn comprises tropoelastin and a biodegradable polymer. In some embodiments of any of the below- or above-mentioned embodiments, the polymer is polycaprolactone. In some embodiments of any of the below- or above-mentioned embodiments, PCL is blended with additional natural polymers.

Polycaprolactone is a biodegradable polyester with a melting point of around about 60° C. and a glass transition temperature of about −60° C. The most common use of polycaprolactone is in the production of specialty polyurethanes. Polycaprolactone is described in the embodiments herein for the methods of making an electrospun fibrous yarn.

Foreign body response, may refer to the biological response to an implant, for example. In the embodiments described herein, the hybrid yarn causes no tissue encapsulation of an implant, or inflammation, for example.

Pelvic organ prolapse as described herein may refer to the weakening of muscles or tissues that support the pelvic organs such as the uterus, bladder, or rectum. In some embodiments described herein, methods are directed to treatment or prevention of pelvic organ prolapse.

Yarn may be described as a fiber-like composition or formulation that is then incorporated into a product, such as a mesh or a tissue engineering scaffold.

The mesh or tissue engineering scaffold disclosed herein may have a Young's modulus of about 5 MPa to about 65 MPa. In some embodiments, the mesh or tissue engineering scaffold has a Young's modulus of about 5 MPa, about 10 MPa, about 15 MPa, about 20 MPa, about 25 MPa, about 30 MPa, about 35 MPa, about 40 MPa, about 45 MPa, about 50 MPa, about 55 MPa, about 60 MPa, or about 65 MPa. In other embodiments, the mesh has a Young's modulus similar to the modulus for a tissue (e.g., vaginal tissue) in which it is implanted (e.g., a Young's modulus within 5%, 10%, 15%, 20%, 25%, 30%, 35%, 40%, or 50% of the Young's modulus of the tissue).

In a preferred embodiment, the mesh or tissue engineering scaffold is implanted into vaginal tissue and has a Young's modulus similar to that of vaginal tissue (e.g., about 30 MPa, about 31 MPa, about 32 MPa, about 33 MPa, about 34 MPa, about 35 MPa, about 36 MPa, about 37 MPa, about 38 MPa, about 39 MPa, or about 40 MPa).

Additionally, the mesh or tissue engineering scaffold may have an ultimate tensile strength (UTS) of about 5 MPa to about 65 MPa. In some embodiments, the mesh or tissue engineering scaffold has a UTS of about 5 MPa, about 10 MPa, about 15 MPa, about 20 MPa, about 25 MPa, about 30 MPa, about 35 MPa, about 40 MPa, about 45 MPa, about 50 MPa, about 55 MPa, about 60 MPa, or about 65 MPa.

The mesh or tissue engineering scaffold may be capable of elongating about 5% to about 200% more than its original length. In some embodiments the mesh or tissue engineering scaffold is capable of elongating about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or greater.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method of making a hybrid material, the method comprising: providing tropoelastin; providing a biodegradable polymer; and mixing the tropoelastin and biodegradable polymer to produce a mixture; wherein the mixture results in a hybrid material.

Clause 2. The method of Clause 1, wherein the biodegradable polymer is polycaprolactone (PCL), poly(lactic acid), poly (lactic-co-glycolic acid, polyglycolic acid, poly(trimethylene carbonate, poly-4-hydroxybutyrate or a co-polymer of any one of the aforementioned polymers.

Clause 3. The method of Clause 1 or 2, wherein the biodegradable polymer is polycaprolactone (PCL).

Clause 4. The method of any one of Clauses 1-3, wherein the tropoelastin is provided as a monomer in solution.

Clause 5. The method of any one of Clauses 1-3, wherein the tropoelastin is provided as tropoelastin particles.

Clause 6. The method of any one of Clauses 1-5, wherein the method further comprises melting the biodegradable polymer after the providing step, thereby producing a molten biodegradable polymer, and suspending the tropoelastin in the molten biodegradable polymer prior to the mixing step.

Clause 7. The method of any one of Clauses 1-5, wherein the method further comprises dissolving the biodegradable polymer and dissolving the tropoelastin prior to the mixing step and mixing the dissolved biodegradable polymer and the dissolved tropoelastin.

Clause 8. The method of any one of Clauses 1-5, wherein the method further comprises dissolving the biodegradable polymer, and suspending the tropoelastin particles in the dissolved biodegradable polymer prior to the mixing step.

Clause 9. The method of any one of Clauses 1-8, wherein the method further comprises printing or casting the mixture.

Clause 10. The method of any one of Clauses 1-9, wherein the hybrid material is a yarn.

Clause 11. The method of any one of Clauses 1-5, wherein the method further comprises electrospinning the mixture, thereby forming an electrospun fibrous yarn.

Clause 12. The method of Clause 11, wherein the method further comprises collecting the electrospun fibrous yarn.

Clause 13. The method of any one of Clauses 1-12, wherein the method further comprises washing the hybrid material.

Clause 14. The method of any one of Clauses 1-13, wherein the mixture comprises a ratio of tropoelastin to biodegradable polymer of about 99:1, about 95:5, about 90:10, about 80:20, about 70:30, about 75:25, about 60:40, about 50:50, about 40:60, about 30:70, about 25:75, about 10:90 or about 0:100.

Clause 15. The method of any one of Clauses 1-14, wherein the mixture comprises a ratio of tropoelastin to biodegradable polymer of about 99:1, about 95:5, about 75:25, about 50:50, about 25:75 or about 0:100.

Clause 16. The method of any one of Clauses 1-15, wherein the mixture comprises a ratio of tropoelastin to biodegradable polymer of about 50:50, about 25:75 or about 0:100.

Clause 17. The method of any one of Clauses 1-16, wherein the mixture comprises a ratio of tropoelastin to biodegradable polymer of about 50:50.

Clause 18. The method of any one of Clauses 1-16, wherein the mixture comprises a ratio of tropoelastin to biodegradable polymer of about 25:75.

Clause 19. The method of any one of Clauses 1-16, wherein the mixture comprises a ratio of tropoelastin to biodegradable polymer of about 0:100.

Clause 20. The method of any one of Clauses 10-19, wherein the yarn or electrospun fibrous yarn comprises a length of about 1 cm, about 5 cm, about 15 cm, 15 cm, about 20 cm, about 25 cm, about 30 cm, about 35 cm, about 40 cm, about 45 cm, about 50 cm, about 75 cm, about 100 cm, about 125 cm, about 150 cm, about 175 cm, about 200 cm, about 225 cm, about 250 cm, about 275 cm, about 300 cm, about 325 cm, about 350 cm, about 375 cm, about 400 cm, about 425 cm, about 450 cm, about 475 cm, about 500 cm, about 525 cm, about 550 cm, about 575 cm, about 600 cm, about 625 cm, about 650 cm, about 675 cm, about 700 cm or any length in between a range defined by any two aforementioned values.

Clause 21. The method of any one of Clauses 1-20, wherein the method is performed at a relative humidity of between about 0% to about 5%, about 5% to about 10%, about 10% to about 15%, about 15% to about 20%, about 20% to about 25%, about 25% to about 30%, about 35% to about 40%, about 35% to about 40%, about 40% to about 45%, about 45% to about 50%, about 50% to about 55%, about 55% to about 60% or about 60% to about 65%.

Clause 22. The method of any one of Clauses 1-20, wherein the method is performed at a relative humidity of between about 35% to about 61%.

Clause 23. The method of any one of Clauses 1-20, wherein the method is performed at a relative humidity of between about 42%-62%.

Clause 24. The method of any one of Clauses 1-23, wherein the ratio of tropoelastin to PCL is about 75:25, about 50:50 or about 25:75.

Clause 25. The method of any one of Clauses 1-16 or 19-23, wherein the ratio of tropoelastin to PCL is about 0:100.

Clause 26. The method of any one of Clauses 11-25, wherein the electrospinning is performed with an electrospinner comprising a funnel collector, wherein the funnel collector comprises a funnel collector speed of about 400 rpm, about 425 rpm, about 450 rpm, about 475 rpm, about 500 rpm, about 525 rpm, about 550 rpm, about 575 rpm, about 600 rpm, about 625 rpm, about 650 rpm, about 675 rpm, about 700 rpm, about 725 rpm, about 750 rpm, about 775 rpm, about 800 rpm, about 825 rpm, about 850 rpm, about 875 rpm, about 900 rpm, about 925 rpm, about 950 rpm, about 975 rpm, about 1000 rpm, or about 1250 rpm or any speed in between a range defined by any two aforementioned values.

Clause 27. The method of Clause 26, wherein the electrospinner further comprises a rotating winder speed, wherein the rotating winder speed comprises a speed of about 2 rpm, about 3 rpm, about 4 rpm, about 5 rpm, about 6 rpm, about 7 rpm, about 8 rpm, about 9 rpm, about 10 rpm, about 11 rpm, about 12 rpm or about 13 rpm or any speed in between a range defined by any two aforementioned values.

Clause 28. The method of Clauses 26 or 27, wherein the funnel collector speed and or rotating winder speed is adjusted depending on the relative humidity.

Clause 29. The method of any one of Clauses 1-28, wherein the mixing step is performed for at least about 4 hours.

Clause 30. The method of any one of Clauses 1-29, wherein the mixing step is performed at about 4° C.

Clause 31. A method of making a hybrid material, the method comprising: providing tropoelastin; providing a biodegradable polymer; melting the biodegradable polymer, thereby producing a melted biodegradable polymer; suspending the tropoelastin into the melted biodegradable polymer; producing a mixture; and printing or casting the mixture; thereby producing a hybrid material.

Clause 32. A method of making a hybrid material, the method comprising: providing tropoelastin; providing a biodegradable polymer; dissolving the tropoelastin; dissolving the biodegradable material; mixing the tropoelastin and biodegradable material thereby producing a mixture; and printing or casting the mixture; thereby producing a hybrid material.

Clause 33. A method of making a hybrid material, the method comprising: providing tropoelastin; providing a biodegradable polymer; dissolving the biodegradable polymer

suspending the tropoelastin into the biodegradable polymer, thereby producing a mixture; and printing or casting the mixture; thereby producing a hybrid material.

Clause 34. The method of any one of Clauses 31-33, wherein the hybrid material is a yarn.

Clause 35. A method of making a hybrid material, the method comprising: providing tropoelastin; providing a biodegradable polymer; mixing the tropoelastin and biomaterial to produce a mixture; electrospinning the mixture; and collecting the hybrid material in a form of an electrospun fibrous yarn.

Clause 36. The method of any one of Clauses 31-35, wherein the tropoelastin is provided as a monomer in solution.

Clause 37. The method of any one of Clauses 31-35, wherein the tropoelastin is provided as tropoelastin particles.

Clause 38. A hybrid material, the material comprising: tropoelastin; and a biodegradable polymer.

Clause 39. The hybrid material of Clause 38, wherein the hybrid material is a casted material.

Clause 40. The hybrid material of Clause 38, wherein the hybrid material is a printed material.

Clause 41. The hybrid material of Clause 38, wherein the hybrid material is a yarn.

Clause 42. The hybrid material of Clause 38, wherein the hybrid material is an electrospun yarn.

Clause 43. The hybrid material of any one of Clauses 38-42, wherein the biodegradable polymer is polycaprolactone (PCL), poly(lactic acid), poly (lactic-co-glycolic acid, polyglycolic acid, poly(trimethylene carbonate, poly-4-hydroxybutyrate or a co-polymer of any one of the aforementioned polymers.

Clause 44. The hybrid material of any one of Clauses 38-43, wherein the biodegradable polymer is polycaprolactone (PCL).

Clause 45. The hybrid material of any one of Clauses 38-44, wherein PCL comprises a molecular weight of about 1,250 g/mol, about 2,500 g/mol, about 3,750 g/mol, about 5,000 g/mol, about 6,250 g/mol, about 7,500 g/mol, about 8,750 g/mol, about 9,000 g/mol, about 10,000 g/mol, about 45,000 g/mol, about 80,000 g/mol, about 90,000 g/mol or about 100,000 g/mol.

Clause 46. The hybrid material of any one of Clauses 38-45, wherein the PCL comprises a molecular weight of about 80,000 g/mol.

Clause 47. The hybrid material of any one of Clauses 38-46, wherein the material comprises a ratio of tropoelastin to biodegradable polymer of about 90:10, about 80:20, about 70:30, about 75:25, about 60:40, about 50:50, about 40:60, about 30:70, about 25:75, about 10:90 or about 0:100.

Clause 48. The hybrid material of any one of Clauses 38-47, wherein the material comprises a ratio of tropoelastin to biodegradable polymer of about 75:25, about 50:50, about 25:75 or about 0:100.

Clause 49. The hybrid material of any one of Clauses 38-48, wherein the material comprises a ratio of tropoelastin to biodegradable polymer of about 50:50, about 25:75 or about 0:100.

Clause 50. The hybrid material of any one of Clauses 38-49, wherein the material comprises a ratio of tropoelastin to biodegradable polymer of about 50:50.

Clause 51. The hybrid material of any one of Clauses 38-49, wherein the material comprises a ratio of tropoelastin to biodegradable polymer of about 25:75.

Clause 52. The hybrid material of any one of Clauses 38-49, wherein the material comprises a ratio of tropoelastin to biodegradable polymer of about 0:100.

Clause 53. The hybrid material of any one of Clauses 38-52, wherein the hybrid material is biocompatible and biodegradable.

Clause 54. The hybrid material of any one of Clauses 38-53, wherein the scaffold is non-toxic, and wherein breakdown products or by-products of the yarn do not interfere with tissue function.

Clause 55. The hybrid material of any one of Clauses 38-54, wherein the tropoelastin is monomeric.

Clause 56. The hybrid material of any one of Clauses 38-55, wherein the tropoelastin is not crosslinked.

Clause 57. The hybrid material of any one of Clauses 38-56, wherein the hybrid material maintains structural integrity following exposure to aqueous solution.

Clause 58. The hybrid material of any one of Clauses 38-57, wherein the hybrid material maintains structural integrity at a temperature of at least about 37° C.

Clause 59. The hybrid material of any one of Clauses 38-58, wherein the hybrid material maintains structural integrity at a temperature of about 37° C.

Clause 60. The hybrid material of any one of Clauses 38-59, wherein the hybrid material supports fibroblast growth.

Clause 61. The hybrid material of Clause 60, wherein fibroblast growth is supported for at least about 7 days.

Clause 62. The hybrid material of any one of Clauses 38-61, wherein the hybrid material has a minimized foreign body response in tissue.

Clause 63. The hybrid material of any one of Clauses 38-62, wherein the hybrid material produces minimal inflammation in tissue.

Clause 64. The hybrid material of any one of Clauses 38-63, wherein the hybrid material is a yarn or an electrospun yarn, wherein the yarn or electrospun yarn comprises a fiber width of about 150 nm, about 200 nm, about 300 nm, 400 nm, about 450 nm, about 500 nm, about 550 nm, about 600 nm, about 650 nm, about 700 nm, about 750 nm, about 800 nm, about 850 nm, about 900 nm, about 1000 nm, about 1050 nm, about 1100 nm, about 1200 nm, about 1400 nm, about 1600 nm, about 1800 nm, about 2000 nm, about 2500 nm, about 3000 nm, about 3500 nm, about 4000 nm, about 4500 nm, about 5000 nm, about 5500 nm, about 6000 nm, about 6500 nm, about 7000 nm, about 7500 nm, about 8000 nm, about 8500 nm, about 9000 nm, about 10,000 nm or any fiber width in between a range defined by any two aforementioned values.

Clause 65. The hybrid material of any one of Clauses 38-64, wherein the hybrid material is a yarn or an electrospun yarn, wherein the yarn or electrospun yarn comprises a fiber twist angle of about 5°, about 10°, about 15°, about 20°, about 25°, about 30°, about 35°, about 40°, about 50°, about 55°, about 60°, about 65°, about 70°, about 75°, about 80°, about 85°, about 90°, about 95° or any angle in between a range defined by any two aforementioned values.

Clause 66. The hybrid material of any one of Clauses 38-65, wherein the hybrid material is a yarn or an electrospun yarn, wherein the yarn or electrospun yarn comprises a yarn width of about 50 μm, about 75 μm, about 100 μm, about 125 μm, about 150 μm, about 175 μm, about 200 μm, about 275 μm, 300 μm, about 325 μm, about 350 μm, about 375 μm, about 400 μm, about 425 μm, about 450 μm, about 475 μm, about 500 μm, about 525 μm, about 500 μm, about 525 μm, about 550 μm, about 575 μm, about 600 μm, about 625 μm, about 650 μm, about 675 μm, about 700 μm, about 725 μm, about 750 μm, about 775 μm, about 800 μm, about 825 μm, about 850 μm, about 875 μm, about 900 μm, about 925 μm, about 950 μm, about 975 μm or any yarn width in between a range defined by any two aforementioned values.

Clause 67. The hybrid material of any one of Clauses 38-66, wherein the biopolymer is absorbable.

Clause 68. A tissue engineering scaffold for tissue repair, the scaffold comprising: a hybrid material, wherein the hybrid material comprises: tropoelastin; and a biodegradable polymer.

Clause 69. The tissue engineering scaffold of Clause 68, wherein the hybrid material is a printed.

Clause 70. The tissue engineering scaffold of Clause 68, wherein the hybrid material is casted.

Clause 71. The tissue engineering scaffold of Clause 68, wherein the hybrid material is a yarn.

Clause 72. The tissue engineering scaffold of Clause 68, wherein the hybrid material is an electrospun yarn.

Clause 73. The tissue engineering scaffold of any one of Clauses 68-72, wherein the biodegradable polymer comprises polycaprolactone (PCL), poly(lactic acid), poly (lactic-co-glycolic acid, polyglycolic acid, poly(trimethylene carbonate, poly-4-hydroxybutyrate or a co-polymer of any one of the aforementioned polymers.

Clause 74. The scaffold of any one of Clauses 68-73, wherein the biodegradable polymer comprises polycaprolactone (PCL).

Clause 75. The scaffold of any one of Clauses 68-74, wherein the scaffold comprises a ratio of tropoelastin to biodegradable polymer of about 90:10, about 80:20, about 70:30, about 75:25, about 60:40, about 50:50, about 40:60, about 30:70, about 25:75, about 10:90 or about 0:100.

Clause 76. The scaffold of any one of Clauses 68-75, wherein the scaffold comprises a ratio of tropoelastin to biodegradable polymer of about 75:25, about 50:50, about 25:75 or about 0:100.

Clause 77. The scaffold of any one of Clauses 68-76, wherein the scaffold is biocompatible and biodegradable.

Clause 78. The scaffold of any one of Clauses 68-77, wherein the scaffold is non-toxic, and wherein breakdown products or by-products of the scaffold do not interfere with tissue function.

Clause 79. The scaffold of any one of Clauses 68-78, wherein the scaffold supports in vitro fibroblast growth.

Clause 80. The scaffold of any one of Clauses 68-79, wherein the in vitro fibroblast growth is supported for at least about 7 days.

Clause 81. The scaffold of any one of Clauses 68-80, wherein the scaffold provides a structure to allow cells to attach and infiltrate.

Clause 82. The scaffold of any one of Clauses 68-81, wherein the scaffold promotes cellular growth and cellular proliferation.

Clause 83. The scaffold of any one of Clauses 68-82, wherein the scaffold provides structural support to cells and promotes repair of tissues by enabling tissues to attach to a surface of the scaffold and enables proliferation.

Clause 84. The scaffold of any one of Clauses 68-83, wherein the scaffold has a low in vivo degradation rate, wherein the degradation is in excess of two weeks or in excess of four weeks.

Clause 85. The scaffold of any one of Clauses 68-84, wherein the scaffold promotes elastogenesis and angiogenesis.

Clause 86. The scaffold of any one of Clauses 68-85, wherein the scaffold does not lead to inflammation of the tissues and does not lead to foreign body response.

Clause 87. The scaffold of any one of Clauses 68-86, wherein the scaffold comprises a hybrid yarn comprised of the tropoelastin and the biodegradable polymer.

Clause 88. The scaffold of any one of Clauses 68-86, wherein the scaffold comprises an electrospun hybrid yarn comprised of the tropoelastin and the biodegradable polymer.

Clause 89. The scaffold of Clause 87 or 88, wherein the scaffold comprises randomly arranged fibers of the hybrid yarn or electrospun hybrid yarn.

Clause 90. The scaffold of any one of Clauses 87 or 89, wherein the scaffold comprises continuous yarns comprising the hybrid yarn or electrospun hybrid yarn, wherein the yarns comprise aligned fibers that are capable of withstanding mechanical stress.

Clause 91. The scaffold of any one of Clauses 68-90, wherein the scaffold allows release of tropoelastin.

Clause 92. A method of tissue repair, the method comprising: providing a tissue engineering scaffold, wherein the tissue engineering scaffold comprises a hybrid yarn, the yarn comprising: tropoelastin; and a biodegradable polymer; and implanting the tissue engineering scaffold into tissue of an individual.

Clause 93. The method of Clause 92, wherein the biodegradable polymer comprises polycaprolactone (PCL), poly(lactic acid), poly (lactic-co-glycolic acid, polyglycolic acid, poly(trimethylene carbonate, poly-4-hydroxybutyrate or a co-polymer of any one of the aforementioned polymers.

Clause 94. The method of Clause 92 or 93, wherein the biodegradable polymer comprises polycaprolactone (PCL), poly(lactic acid).

Clause 95. The method of any one of Clauses 92-94, wherein the scaffold releases monomeric tropoelastin into the tissue of the individual.

Clause 96. The method of any one of Clauses 92-95, wherein the scaffold comprises a ratio of tropoelastin to biodegradable polymer of about 75:25, about 50:50, about 25:75 or about 0:100.

Clause 97. The method of any one Clauses 92-96, wherein the scaffold comprises a ratio of tropoelastin to biodegradable polymer of about 50:50 or about 25:75.

Clause 98. The method of any one of Clauses 92-97, wherein the method promotes synthesis of new elastin in the tissue.

Clause 99. The method of any one of Clauses 92-98, wherein the method is performed for abdominal wall repair.

Clause 100. The method of any one of Clauses 92-98, wherein the method is performed for treating a hernia.

Clause 101. The method of any one of Clauses 92-98, wherein the tissue is vaginal tissue.

Clause 102. The method of Clause 101, wherein the scaffold has a Young's modulus similar to the Young's modulus of vaginal tissue.

Clause 103. The method of Clause 102, wherein the scaffold has a Young's modulus of about 30 MPa, about 31 MPa, about 32 MPa, about 33 MPa, about 34 MPa, about 35 MPa, about 36 MPa, about 37 MPa, about 38 MPa, about 39 MPa, or about 40 MPa.

Clause 104. A breast surgery procedure using the scaffold of any one of Clauses 68-91.

Clause 105. The surgery procedure of Clause 104, wherein the breast surgery procedure is a reconstruction surgery.

Clause 106. The surgery procedure of Clause 104 or 105, wherein the breast surgery procedure further comprises tissue expansion and/or a tissue expander.

Clause 107. The surgery procedure of any one of Clauses 104-105, wherein the breast surgery procedure comprises a vascular flap reconstruction.

Clause 108. The surgery procedure of any one of Clauses 104-107, wherein the breast surgery procedure comprises a breast augmentation with breast implants.

Clause 109. The surgery procedure of any one of Clauses 104-108, wherein the scaffold supports one or a combination of a breast implant or breast tissue when used in reconstructive surgery.

Clause 110. A method of treating pelvic organ prolapse in an individual, the method comprising: providing tissue engineering scaffold, wherein the tissue engineering scaffold comprises a hybrid material, the hybrid material comprising: tropoelastin and PCL in a ratio of tropoelastin to PCL of about 25:75; placing the scaffold into vaginal tissue of the individual.

Clause 111. The method of Clause 110, wherein the hybrid material comprises an electrospun hybrid yarn.

Clause 112. The method of Clause 110 or 111, wherein the method promotes deposition of collagen into the tissue of the individual.

Clause 113. The method of any one of Clauses 110-112, wherein the method promotes deposition of collagen around the scaffold.

Clause 114. The method of any one of Clauses 110-113, wherein the method promotes an anti-inflammatory effect in the tissue surrounding the scaffold.

Clause 115. The method of any one of Clauses 110-114, wherein the method promotes localization of macrophages at an interface between the scaffold and the tissue.

Clause 116. The method of any one of Clauses 110-115, wherein the method promotes tissue regeneration.

Clause 117. The method of any one of Clauses 110-116, wherein the pelvic organ prolapse is caused by a dropped bladder (cystocele).

Clause 118. The method of any one of Clauses 110-117, wherein the pelvic organ prolapse is caused by rectocele.

Clause 119. The method of any one of Clauses 110-117, wherein the pelvic organ prolapse is caused by a dropped uterus (uterine prolapse).

Clause 120. A mesh comprising a yarn, wherein the yarn comprises tropoelastin and a biodegradable polymer.

Clause 121. The mesh of Clause 120, wherein the biodegradable polymer is polycaprolactone (PCL), poly(lactic acid), poly (lactic-co-glycolic acid, polyglycolic acid, poly(trimethylene carbonate, poly-4-hydroxybutyrate or a co-polymer of any one of the aforementioned polymers.

Clause 122. The mesh of Clauses 120-121, wherein the biodegradable polymer is polycaprolactone (PCL).

Clause 123. The mesh of any one of Clauses 120-122, wherein PCL comprises a molecular weight of about 1,250 g/mol, about 2,500 g/mol, about 3,750 g/mol, about 5,000 g/mol, about 6,250 g/mol, about 7,500 g/mol, about 8,750 g/mol, about 9,000 g/mol, about 10,000 g/mol, about 45,000 g/mol, about 80,000 g/mol, about 90,000 g/mol or about 100,000 g/mol.

Clause 124. The mesh of any one of Clauses 122-123, wherein the PCL comprises a molecular weight of about 80,000 g/mol.

Clause 125. The mesh of any one of Clauses 120-124, wherein the mesh comprises a ratio of tropoelastin to biodegradable polymer of about 90:10, about 80:20, about 70:30, about 75:25, about 60:40, about 50:50, about 40:60, about 30:70, about 25:75, about 10:90 or about 0:100.

Clause 126. The mesh of any one of Clauses 120-125, wherein the mesh comprises a ratio of tropoelastin to biodegradable polymer of about 75:25, about 50:50, about 25:75 or about 0:100.

Clause 127. The mesh of any one of Clauses 120-126, wherein the mesh comprises a ratio of tropoelastin to biodegradable polymer of about 50:50, about 25:75 or about 0:100.

Clause 128. The mesh of any one of Clauses 120-127, wherein the mesh comprises a ratio of tropoelastin to biodegradable polymer of about 50:50.

Clause 129. The mesh of any one of Clauses 120-128, wherein the mesh comprises a ratio of tropoelastin to biodegradable polymer of about 25:75.

Clause 130. The mesh of any one of Clauses 120-129, wherein the mesh comprises a ratio of tropoelastin to biodegradable polymer of about 0:100.

Clause 131. The mesh of any one of Clauses 120-130, wherein the mesh is biocompatible and biodegradable.

Clause 132. The mesh of any one of Clauses 120-131, wherein the tropoelastin is monomeric.

Clause 133. The mesh of any one of Clauses 120-132, wherein the tropoelastin is not crosslinked.

Clause 134. The mesh of any one of Clauses 120-133, wherein the mesh maintains structural integrity following exposure to aqueous solution.

Clause 135. The mesh of any one of Clauses 120-134, wherein the mesh maintains structural integrity at a temperature of at least about 37° C.

Clause 136. The mesh of any one of Clauses 120-135, wherein the mesh maintains structural integrity at a temperature of about 37° C.

Clause 137. The mesh of any one of Clauses 120-136, wherein the mesh supports fibroblast growth.

Clause 138. The mesh of Clause 137, wherein fibroblast growth is supported for at least about 7 days.

Clause 139. The mesh of any one of Clauses 120-138, wherein the mesh has a minimized foreign body response in tissue.

Clause 140. The mesh of any one of Clauses 120-139, wherein the mesh produces minimal inflammation in tissue.

### EXAMPLES

The examples disclosed herein are discussed to illustrate application of the disclosure and should not be construed as limiting the disclosure in any way.

### Example 1: Methods of Making the Hybrid Yarn

Materials and Methods

Preparation of Solutions

Four blends of tropoelastin and PCL (Mw=80,000 g/mol) (Sigma-Aldrich, USA) were prepared by dissolving tropoelastin and PCL separately in hexafluoroisopropanol (Sigma-Aldrich, USA) to make a 10% (w/v) solution. Solutions were left for 18 hours at 4° C. and then mixed together for 4 hours on a rotating platform (Ratek, Australia).

Electrospinning of Hybrid Yarn.

An electrospinning apparatus was set up similar to that described by Ali et al. (Journal of the Textile Institute (2012) 103 (1), 80 incorporated by reference in its entirety herein). Electrospinning parameters for this study were based on parameters for the fabrication of tropoelastin:silk hybrid yarns, as defined previously by the Weiss Group (Aghaei-Ghareh-Bolagh et al. “Development of elastic biomaterials as high performance candidates for tissue engineering applications.” University of Sydney (2018); incorporated by reference in its entirety). Two 1 mL syringes were loaded with tropoelastin:PCL solution (10% w/v in hexafluoroisopropanol) and positioned facing a rotating funnel collector. The tropoelastin:PCL solution was pumped through 18-gauge needles which were connected to a 10 kV negative power supply and a 10 kV positive power supply. As the charged polymer fibers deposited on the rotating funnel collector, they were coaxed into forming a fibrous cone through the use of a plastic pipette. A fibrous yarn was withdrawn from the fibrous cone and collected around a rotating winder.

In some embodiments of any of the below- or above-mentioned embodiments, the hybrid material or scaffold may be sterilized. Those of skill in the art would appreciate that there are multiple techniques for sterilizing the hybrid material that does not compromise the function or structure of the hybrid material. In some embodiments of any of the below- or above-mentioned embodiments, the hybrid material or scaffold may be sterilized by radiation. In some embodiments of any of the below- or above-mentioned embodiments, the hybrid material or scaffold may be sterilized by washing in absolute ethanol.

Structural Characterization

Scanning electron microscopy (SEM) was used to characterize tropoelastin:PCL electrospun yarns. Yarns were mounted with silver conductive paint and sputter coated with 15 nm gold. SEM images were collected for measurements using a JEOL Neoscope Tabletop SEM (JEOL, Japan) and fiber width, yarn width and fiber angle were measured using Image J software version 1.52a (National Institutes of Health, USA). Yarns were immersed in Milli-Q water (MQW) and incubated at 37° C., 20° C. or 4° C. for 24 hours. Yarns were then rinsed 3× with MQW and dried overnight at 37° C. Yarns were mounted with silver conductive paint and sputter coated with 15 nm gold. Following water treatments, SEM images were collected using a Zeiss Sigma HD FEG SEM (Zeiss, France).

Characterization of Chemical Composition

Fourier transform infrared spectroscopy (FTIR) was performed on a Bruker LUMOS FTIR Microscope spectrometer (Bruker, USA) fitted with a micro-ATR pressure-controlled crystal. For each measurement, 64 scans were averaged with a 4 cm-1 resolution using medium pressure. Spectral analysis was performed with OPUS software version 7.5 (Cooperative Library Network Berlin-Brandenburg, Germany). Atmospheric compensation and baseline correction were applied to all spectra.

Stability

Stability in Phosphate Buffered Saline (PBS) was investigated by weighing yarns and then immersing in PBS. Yarns were incubated at 37° C., 20° C. and 4° C. Protein released was qualitatively assessed using Sodium Dodecyl Sulphate Polyacrylamide Gel Electrophoresis (SDS-PAGE) to confirm if tropoelastin was released from tropoelastin:PCL yarns. Loading buffer (4×) (Life Technologies, USA) was added to protein samples. The samples were then heat denatured at 95° C. for 6 minutes. Samples and Mark12™ unstained protein standard (Life Technologies, USA) were loaded onto a 4-12% NuPAGE™ Bis-Tris gel (Life Technologies, USA) and run at 200 V for 35 minutes in NuPAGE™ MES SDS running buffer (Life Technologies, USA). The gel was then fixed with 50% (v/v) methanol for 30 minutes and then stained with Coomassie stain solution for 1 hour. The gel was destained with 25% (v/v) methanol and 10% (v/v) acetic acid for 1 hour. A NanoDrop™ 2000c UV-visible spectrophotometer (Thermo Fisher Scientific, USA) was used to measure protein released by yarns after immersion in PBS. NanoDrop™ was blanked using PBS. Each sample was loaded onto pedestal and absorbance was measured at 280 nm. Protein released was measured at 1, 3, 5 and 7 days.

Cell Culture and Histological Staining

Human dermal fibroblasts (GM3348, Coriell Institute, USA) were cultured in Dulbecco's Modified Eagle's Medium (DMEM, Life Technologies, USA), supplemented with 10% Fetal Bovine Serum (FBS, Life Technologies, USA) and 1% penicillin-streptomycin (Life Technologies, USA). Cells were incubated at 37° C. and 5% CO2. For each tropoelastin:PCL blend, five yarns were aligned and mounted into a 24-well plate crown insert (Sigma-Aldrich, USA), and then sterilized in absolute ethanol (Ajax Finechem, Australia) for 10 minutes. Cells were seeded at a density of 2.5×104 fibroblasts onto tropoelastin:PCL yarns and grown for 7 days. Cell culture media was aspirated and replaced with fresh culture media after 24 hours, and then every 48 hours following this. At day 7, cell culture media was removed from each well and fibroblasts and yarns were washed 3× with PBS. Fibroblasts and yarns were fixed with 10% formalin (Sigma-Aldrich, USA) for 24 hours at room temperature and then washed 3× with PBS. Triton™ X-100 0.2% (Sigma-Aldrich, USA) was added to cells and yarns for 6 minutes and then rinsed 3× with PBS. Cells were stained with ActinRed™ 555 ReadyProbes (Thermo Fisher Scientific, USA) to stain F-actin, and TO-PRO™ 3 iodide (Thermo Fisher Scientific, USA) to stain nucleus for 30 minutes protected from light. Fibroblasts and yarns were washed 3× with PBS and then confocal images were collected using a Nikon Ti-E Spinning Disk microscope (Nikon, Japan). Excitation/emission wavelengths were 540/565 nm and 642/661 nm for ActinRed™ 555 ReadyProbes and TO-PRO™ 3 iodide staining, respectively.

### Example 2: PCL Mesh for In Vivo Studies

Fabrication of Tropoelastin:PCL Mesh for In Vivo Study

Surgical Implantation of Tropoelastin:PCL Mesh into Ovine Vagina

Two multiparous Border Leicester Merino (BLM) ewes which had delivered lambs at least 3 times were chosen, one to evaluate the Tropoelastin:PCL mesh in this study, the second an incision control. Anesthesia was induced by intravenous Medetomidine premedication (0.1-0.2 mg/kg) followed by intravenous Thiopentone (10 mg/kg), and then maintained with Isoflurane (1-3% in 100% O2). Pain relief was provided before start of surgery as Fentanyl (75 μg/hr) transdermal patch and Carprofen (2 mg/kg) given subcutaneously. A short acting broad-spectrum antibiotic, Cefazolin (7.5 mg/kg), was given intravenously prior to surgery, and a long-acting antibiotic, Duplocilin (5.75 mg/kg), to continue coverage for 48 hours post-surgery. Ewes were placed into lithotomy position. Hydrodissection of the vaginal tissue layers was with 20 ml of bupivacaine (5 mg/ml) with 1 ml of adrenaline (Aspen Pharmacare Australia, 1 mg/ml). A 40 mm, full-thickness midline incision was made on the posterior vaginal wall and the rectovaginal space was dissected. A 3×2 cm Tropoelastin:PCL mesh was surgically implanted and fixed with absorbable sutures into the vaginal wall, and the vaginal epithelium closed using absorbable sutures. Additional pain relief was bupivacaine (5 mg/ml) given subcutaneously at the incision site at end of surgery.

Post Mortem and Histological Analysis of Ovine Vaginal Tissue

Ewes were euthanized after 30 days using Lethabarb (110 mg/kg, Virbac (Australia) and the whole vaginal tract was explanted, trimmed and tissue areas with scaffolds were identified, dissected and fixed using 10% formalin and 4% paraformaldehyde and embedded into paraffin and frozen blocks, respectively.

Paraffin blocks were sectioned at 8 μm and stained with hematoxylin and eosin (H&E), Gomori Trichome, Picro Sims red and Verhoff Van Gieson collagen and elastin stains in the Monash Histology Platform (MHP) using previously published methods. Images were obtained by Aperio scanning or using an Olympus BX61 light microscope.

Immunohistochemical staining was performed on FFPE sections following antigen retrieval using 0.1 M citrate buffer, blocking endogenous peroxidase with 3% H2O2, incubation with protein block (Dako) for 30 min at RT using mouse anti-CD45 (0.5 μg/mL, BioRad) and mouse anti-CD206 (0.5 μg/Ml, Dendritics), primary antibodies for 1 h at 37 C as previously published. Isotype matched IgG antibodies at the same concentration were used as negative controls. HRP-labelled polymer (Dako) conjugated anti-mouse secondary antibody was applied for 40 mins at RT and DAB chromogen (Sigma-Aldrich).

Immunofluorescence staining was performed on PFA-fixed cryosections blocked with protein block using mouse anti-CD45 and rat anti-CD206 and incubated for 1 h at RT. Anti-mouse conjugated Alexa Fluor™-488 and anti-rat conjugated Alexa Fluor™ 568 secondary antibodies (both Thermo-Fisher) were then incubated for 30 min at RT. Nuclei were stained with Hoechst 33258 (Molecular Probes) for 5 minutes. For Collagen III immunofluorescence antigen retrieval was with 0.1% Triton X for 90 s, then protein block was applied followed by rabbit anti Collagen III alpha 1 (1/50, Novus) for 1 h at RT and Alexa-488 anti-rabbit secondary antibody and Hoechst 33258. Images were captured using a FV1200 confocal microscope.

Statistical Analysis

Data presented is expressed as mean±standard deviation and analyzed using one-way or two-way analysis of variance (ANOVA) using GraphPad Prism version 7.0b software (GraphPad Software, USA). Tukey's multiple comparison test was used to determine significant difference between different conditions. Data was statistically significant when p<0.05. Significant difference is indicated in figures as *=p<0.01, **=p<0.001, ***=p<0.001. ns=no significance.

Electrospinning

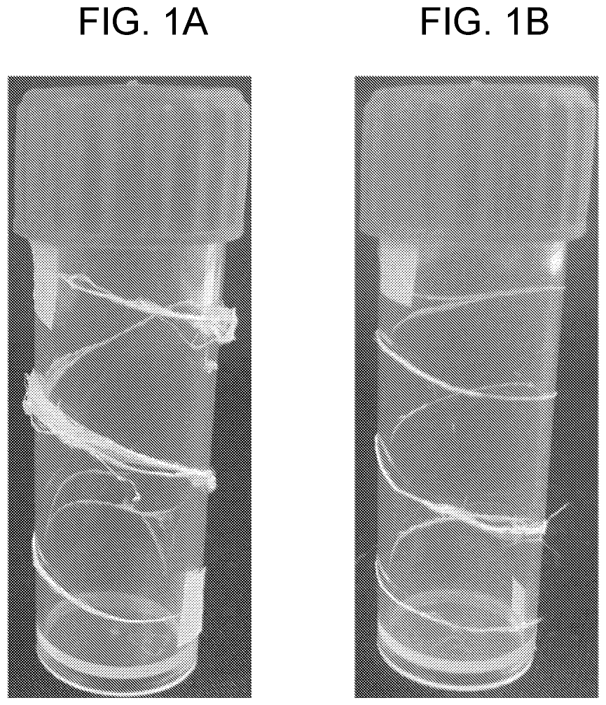

Tropoelastin:PCL hybrid electrospun yarns were fabricated by using an electrospinner set up similar to that described by Ali et al. Parameters defined previously by the Weiss Group formed the foundation of initial electrospinner set up (Aghaei-Ghareh-Bolagh et al. 2018; incorporated by reference herein). Tropoelastin:PCL electrospun yarns fabricated using initial parameters were sometimes poorly formed and not homogeneous in width (FIG. 1A). The electrospinner was set up in a laboratory with a consistent temperature, however relative humidity levels were constantly changing. It was necessary to adjust the funnel speed and winder speed (rpm) to successfully fabricate continuous tropoelastin:PCL yarns as environmental conditions in the laboratory changed (Table 1).

TABLE 1
Longest continuous tropoelastin:PCL electrospun yarns fabricated
with adjusted funnel collector speed and rotating winder speed.

| | Longest | | Funnel | Rotating |
|---|---|---|---|---|
| | yarns | Relative | collector | winder |
| | produced | humidity | speed | speed |
| Tropoelastin:PCL | (cm) | (%) | (rpm) | (rpm) |
| 75:25 | 118 | 40-42 | 750 | 10 |
| | 86 | 53-55 | 1000 | 10 |
| | 69 | 40-44 | 750 | 10 |
| 50:50 | 621 | 45-51 | 875-1000 | 10 |
| | 445 | 46-48 | 875 | 10 |
| | 157 | 56 | 1000 | 10 |
| 25:75 | 290 | 38-39 | 775 | 9 |
| | 286 | 36-38 | 775 | 8 |
| | 182 | 48 | 1000 | 10 |
| 0:100 | 141 | 55 | 1000 | 10 |
| | 113 | 52 | 1000 | 10 |
| | 86 | 53-55 | 1000 | 10 |

The longest continuous 75:25, 50:50 and 25:75 tropoelastin:PCL yarns were fabricated in relative humidity levels between 36-55%, with an adjusted rotating funnel collector speed of between 750-875 rpm for relative humidity levels between 36-48%, or 1000 rpm for relative humidity levels 48% and above. The rotating winder speed was adjusted to 8 rpm when relative humidity was 36-38%, or 9 rpm when relative humidity was 38-39%. The longest 0:100 yarns were produced with a funnel collector speed of 1000 rpm for relative humidity 52-55%. 50:50 and 25:75 tropoelastin:PCL yarns were consistently capable of being produced in multi-meter lengths. The longest 50:50 yarn was 621 cm in length, and the longest 25:75 yarn was 290 cm.

TABLE 2
Working relative humidity levels for successful fabrication
of continuous tropoelastin:PCL electrospun yarns.

| Tropoelastin:PCL | Relative humidity (%) |
|---|---|
| 75:25, 50:50, 25:75 | 35-61 |
| 0:100 | 42-62 |

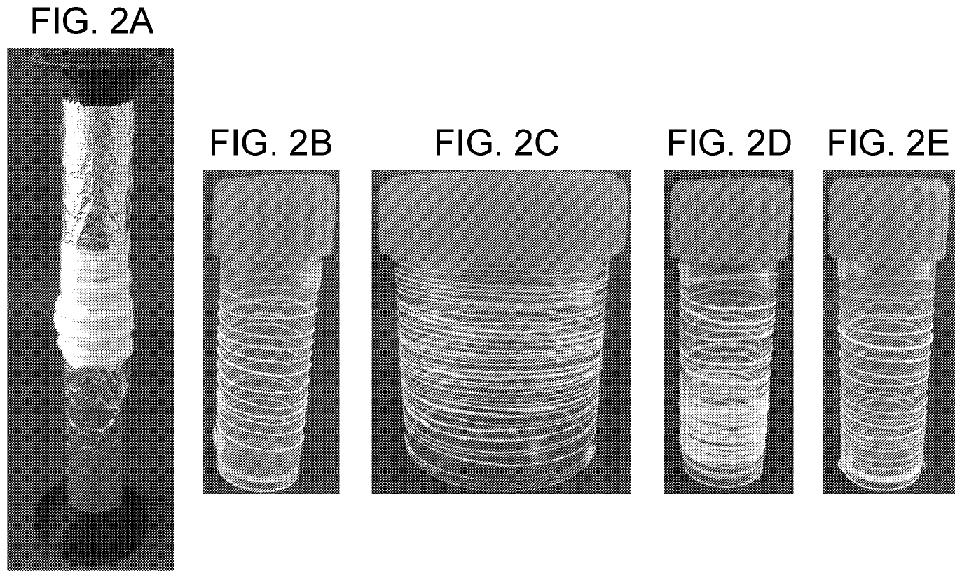

Relative humidity was required to be between 35-61% to successfully fabricate continuous 75:25, 50:50, and 25:75 tropoelastin:PCL yarns, or 42-62% for 0:100 yarns (Table 2). Despite using adjusted rotating funnel collector and winder speeds, relative humidity below these levels produced loosely twisted yarns for blends that incorporated tropoelastin, or brittle 0:100 yarns, which were difficult to handle without causing breakage (FIG. 1B). There was reduced fiber deposition on the rotating funnel collector when electrospinning in relative humidity levels above 61% for 75:25, 50:50 or 25:75 blends or 62% for 0:100 tropoelastin:PCL. Homogeneous 75:25, 50:50, 25:75 and 0:100 tropoelastin:PCL yarns were consistently fabricated through the use of optimum electrospinning settings within the working relative humidity range (FIGS. 2A-2E). The ability to fabricate homogeneous yarns of multi-meter lengths is of importance as they can be woven into more complex structures, such as mesh constructs for surgical use (Wu et al. 2017).

Structural Characterization

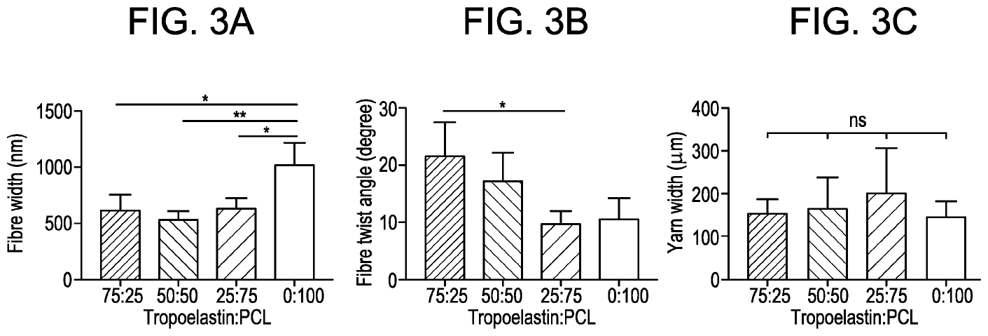

Scanning electron microscopy (SEM) confirmed the tropoelastin:PCL electrospun yarns were fibrous. Tropoelastin:PCL fibers in 0:100 yarns were 1026±186 nm in width (FIG. 3A). The widths of these fibers in this study are in agreement with previous studies of electrospun PCL fiber widths (Chen et al. Tissue Eng (2007) 13 (3), 579, Kim et al. Journal of Materials Science: Materials in Medicine (2013) 24 (6), 1425; incorporated by reference in its entirety herein). 0:100 fibers were significantly wider than fibers containing tropoelastin, whereby 75:25 fibers were 618±144 nm in width, 50:50 and 25:75 nanofibers were 541±70 nm and 645±75 nm, respectively. SEM micrographs revealed the fibers aligned together to form a twist in the yarn (FIG. 3B). 75:25 nanofibers had a fiber twist angle of 22±6°, which was significantly larger than 25:75 nanofibers, which had a twist angle of 10±2°. A larger twist angle has been reported to increase flexibility and tensile strength (Ali et al. 2012), however further testing will need to confirm this, as physical properties of tropoelastin and PCL may also influence these factors. There was no significant difference in yarn width between the four different blends of tropoelastin:PCL yarns (FIG. 3C), confirming tropoelastin:PCL yarns produced using this method are homogenous in width.

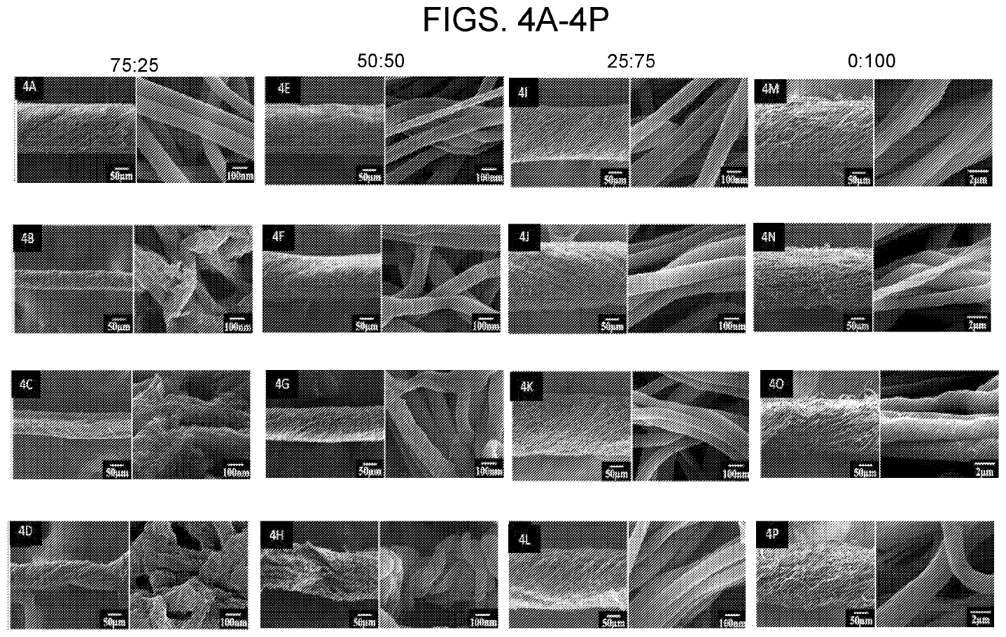

SEM images of each tropoelastin:PCL blend before and after water treatment were collected to assess structural changes. Microscale SEM images of 75:25 tropoelastin yarns showed rounded yarns with visible nanofibers aligned to form a twist (FIG. 4A). Nanoscale SEM images showed separately formed nanofibers with a smooth surface and no obvious signs of wrinkles or craters. After immersion in water at 37° C. for 24 hours (FIG. 4B), the yarn appeared thinner in width and nanofibers were fused together. Nanofibers were no longer smooth and exhibited formation of structures on the surface. Following immersion in water at 20° C. (FIG. 4C), the yarn appeared thinner and flatter in shape. Fusing of nanofibers was evident in nanoscale images after incubation at 20° C. and 4° C. These nanofibers also displayed crater-like structures on the surface. Microscale images following incubation at 4° C. revealed the yarn appeared thinner than the untreated control (FIG. 4D).

Microscale images of untreated 50:50 tropoelastin:PCL revealed yarns were rounded and nanofibrous (FIG. 4E). Minimal wrinkling was observed on nanofibers. Nanofibers displayed a wrinkled appearance after incubation in water across all three temperatures (FIGS. 4F-4H), however after incubation at 4° C., nanofibers changed to a twisted alignment and appeared fused together (FIG. 4H). Microscale images revealed the yarns remained rounded after 37° C. and 20° C. treatments (FIGS. 4B-4C), however after 4° C. incubation, the yarn surface appeared less rounded and uniform, and the nanofibers appear less aligned than the untreated control (FIGS. 4H, 4E).

There was no obvious change in surface structure in 25:75 tropoelastin:PCL yarns before and after water treatment at all three temperatures, both on the microscale and nanoscale (FIGS. 4I-4L). All yarns appeared rounded with single, smooth nanofibers.

Electrospun 0:100 tropoelastin:PCL yarns had no observable difference in all images before and after each treatment (FIGS. 4M-4P). Yarns appeared rounded, however the fibers look not as tightly bundled together compared to 75:25, 50:50 and 25:75 untreated yarns (FIGS. 4A, 4E and 4I).

Mechanical Characterization

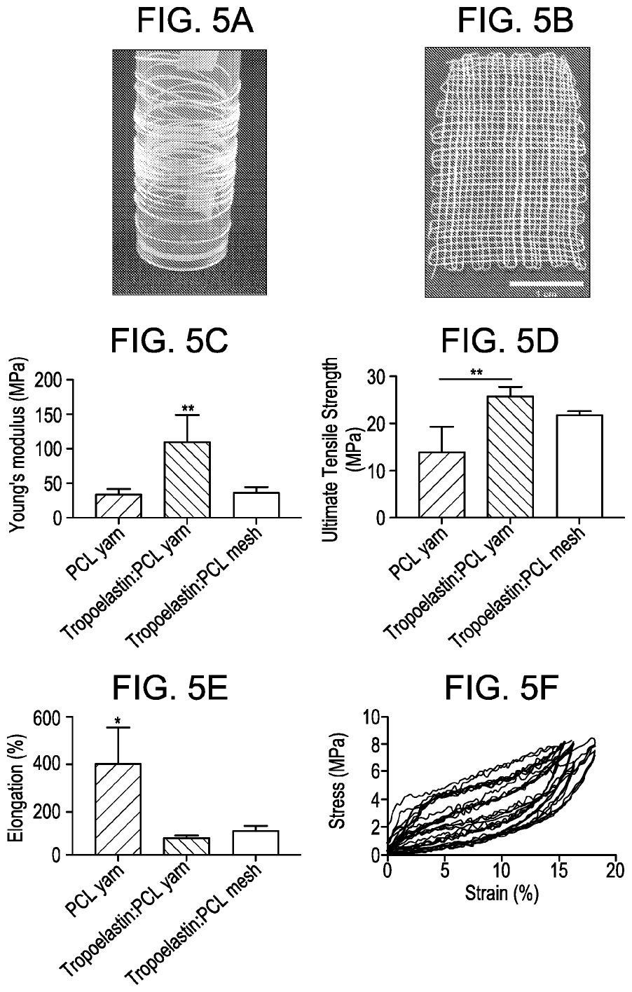

Meshes were woven using 50:50 tropoelastin-PCL yarn (FIGS. 5A and 5B). Yarn and meshes showed similar mechanical properties, except that the meshes had a lower initial Young's modulus than yarns. Without wishing to be bound by a theory of the disclosure, it is hypothesized that the lower initial Young's modulus is a consequence of the free movement of warp over the weft. These meshes had a Young's modulus of 36.5±8.5 MPa which is close to the modulus of 34.3±13.0 MPa reported for ovine vaginal tissue, allowing for mechanical harmony in the tissue of the ovine POP model. The ultimate tensile strength (UTS) and percent elongation of the meshes were 21.8±0.8 MPa and 101±19% respectively (FIGS. 5C, 5D, and 5E).

Tropoelastin:PCL meshes displayed high hysteresis of 49.1±7.7% under cyclic tensile testing (FIG. 5F), However, meshes recovered after each cycle and displayed stable behavior where the cyclic curves of all cycles, except for the first cycle, overlaid. Thus, the meshes are suitable for implantation with non-permanent deformation under comparable strain conditions.

Characterization of Chemical Composition

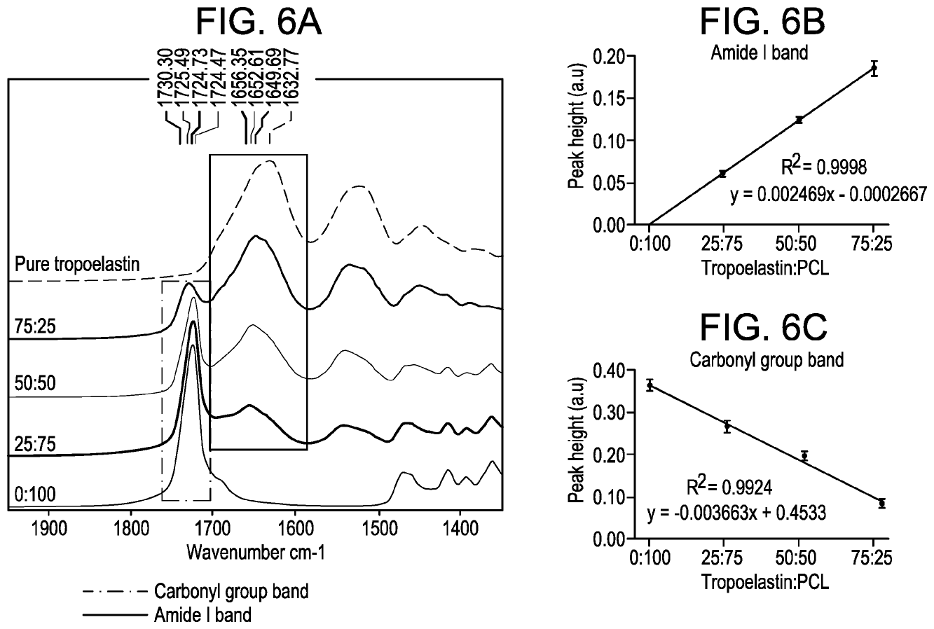

FTIR-ATR analysis was used to characterize the surface chemical composition of each different blend of electrospun tropoelastin:PCL yarn. FTIR-ATR spectra revealed changes between different blends of tropoelastin:PCL (shown as offset spectra in FIG. 6A). For the purpose of this study, one region of each spectra of tropoelastin and PCL were analyzed. The first region is the carbonyl group band (˜1724-1730 cm-1, shaded red), which can be attributed to the stretching vibration of the C═O bond in PCL (Kim et al. Journal of Materials Science: Materials in Medicine (2013) 24 (6), 1425; incorporated by reference in its entirety herein). Comparison of spectra showed the carbonyl group band was absent in pure tropoelastin spectra (grey spectra). The peak height of carbonyl group band decreased as the amount of PCL in each yarn decreased. The Amide I band (˜1632-1656 cm-1, shaded blue), which is the most studied protein band in FTIR-ATR spectra (Hans et al. Journal of Molecular Catalysis B: Enzymatic (1999) 7 (1-4), 207; incorporated by reference in its entirety herein), was absent in 0:100 tropoelastin:PCL spectra (blue spectra), thus confirming tropoelastin was not present in this blend. Amide I band peak height also decreased as the amount of tropoelastin in each yarn decreased. The relationship between peak height and concentration of tropoelastin and PCL in each tropoelastin:PCL blend is shown in FIGS. 6B and 6C respectively. The correlation of determination for Amide I band and carbonyl group band was R2=0.9998 and R2=0.9924 respectively, therefore, variation in Amide I band and carbonyl group band peak heights were due to the amount of tropoelastin and PCL added to polymer mixtures prior to electrospinning, thus confirming the amount of tropoelastin and PCL in each blend is correct.

Stability

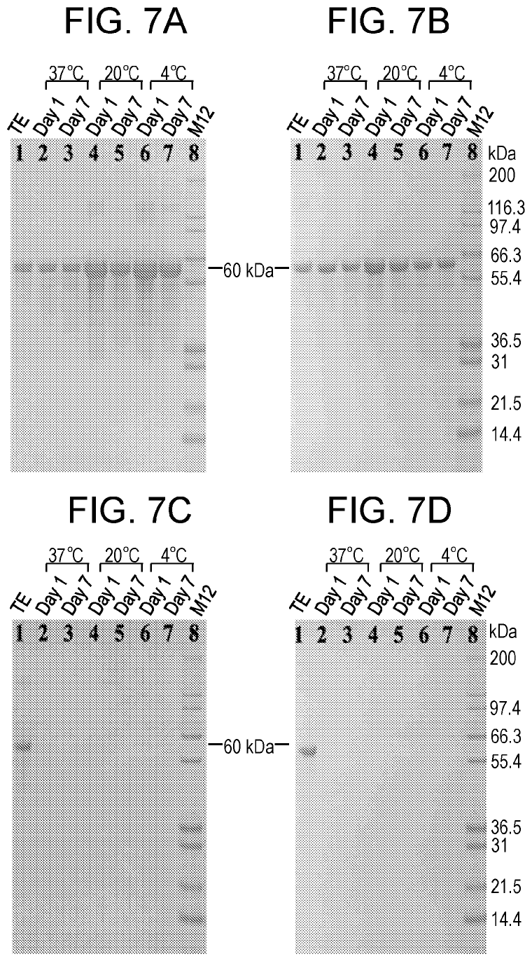

SDS-PAGE was used to confirm tropoelastin was released from the yarns when incubated in PBS. Tropoelastin monomer, at approximately 60 kDa, can be seen in lanes 2-7 in gels FIGS. 7A and 7B, confirming tropoelastin was released from 75:25 and 50:50 tropoelastin:PCL yarns. Less obvious bands were present in the gel corresponding to samples from 25:75 yarns that were incubated at 4° C. (lanes 6 and 7; FIG. 7C). No tropoelastin monomer was evident in any samples from 0:100 yarns (FIG. 7D). The release of tropoelastin from 75:25, 50:50 and 25:75 yarns confirmed the structural changes seen in SEM images following immersion in water can be attributed to loss of tropoelastin (FIGS. 4B, 4D, 4F and 4H). The leaching of tropoelastin was to be expected as it is soluble in water and no cross-linking agent was used to stabilize tropoelastin. Although cross-linking would have retained tropoelastin within yarns for a longer period of time, an initial release of tropoelastin may be beneficial, whereby the presence of tropoelastin in in vitro media promotes elastogenesis by fibroblasts, and the release of tropoelastin from tissue engineering scaffolds has been proven to be pro-angiogenic in vivo (Nivison-Smith et al. Acta Biomater (2010) 6 (2), 354; Mithieux et al. Acta Biomater (2017) 52, 33; Wang et al. Advanced Healthcare Materials (2015) 4 (4), 577; incorporated by reference in their entirety herein).

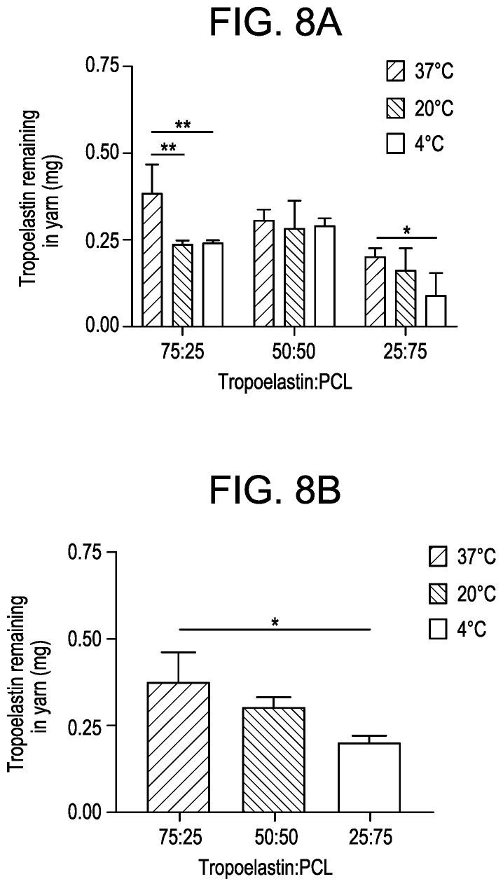

The amount of tropoelastin retained in yarns was determined based on the amount of protein released in PBS at day 7 detected using UV-visible spectroscopy. The results revealed there was 0.39±0.08 mg of tropoelastin remaining in 75:25 yarns after incubation at 37° C. (FIG. 8A), which was significantly more than tropoelastin remaining after incubation at 20° C. (0.24±0.01 mg) or incubation at 4° C. (0.24±0.004 mg). There was no significant difference in tropoelastin remaining in 50:50 yarns across all three temperatures. 25:75 yarns incubated at 37° C. had 0.20±0.02 mg of tropoelastin retained in yarns after 7 days, which was significantly more than 25:75 yarns incubated at 4° C. (0.09±0.06 mg). 75:25 and 25:75 yarns retained more tropoelastin at 37° C. as tropoelastin is more soluble at lower temperatures (Vrhovski et al. European Journal of Biochemistry (1997) 250, 92; incorporated by reference in its entirety herein).

The amount of tropoelastin remaining in yarns after incubation in PBS at 37° C. for 7 days was of interest as these conditions simulate the physiological environment for in vitro studies. 75:25 tropoelastin:PCL yarns had significantly more tropoelastin remaining compared to 25:75 yarns (FIG. 8B). It may be expected tropoelastin retained within the yarns would continue to release as the PCL degrades, however this would need to be confirmed in future studies. Further research will be required to determine long-term degradation rates as each blend of tropoelastin:PCL will degrade at different rates. Consequently, these biomaterials can be tailored to degrade at the optimum rate for its intended application.

Cell Culture and Histological Staining

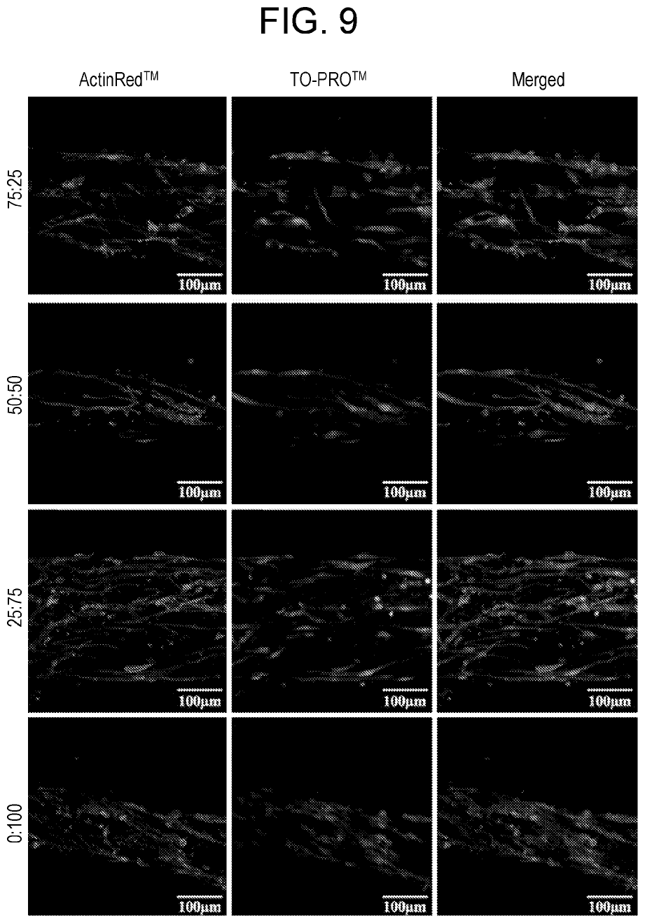

For a biomaterial to be successful, it must be able to support cellular growth. In this study, tropoelastin:PCL hybrid electrospun yarns were seeded with human dermal fibroblasts and then imaged after 7 days. Confocal images confirmed each blend of tropoelastin:PCL electrospun yarn supported human dermal fibroblast growth (FIGS. 9A-9C). Fibroblasts cultured on 75:25, 50:50 and 25:75 yarns appeared larger and elongated compared to fibroblasts growing on 0:100 tropoelastin:PCL yarns. This is likely due to different structural and biological properties of the yarns that incorporate tropoelastin. SEM characterization (FIG. 3A) confirmed tropoelastin blend nanofibers were significantly thinner than 0:100 tropoelastin:PCL microfibers. Nanofibers display increased cell growth compared to microfibers (Chen et al. 2007). Furthermore, PCL, being a synthetic polymer does not provide sites for cellular attachment (Zhang et al. 2005). Tropoelastin is cell interactive and fibroblasts attach to tropoelastin by integrin-mediated adhesion to the C-terminus region of tropoelastin (Bax et al. 2009), thus allowing for fibroblasts to attach and spread on the 75:25, 50:50 and 25:75 scaffolds.

Biomaterial Integration in Sheep Vagina

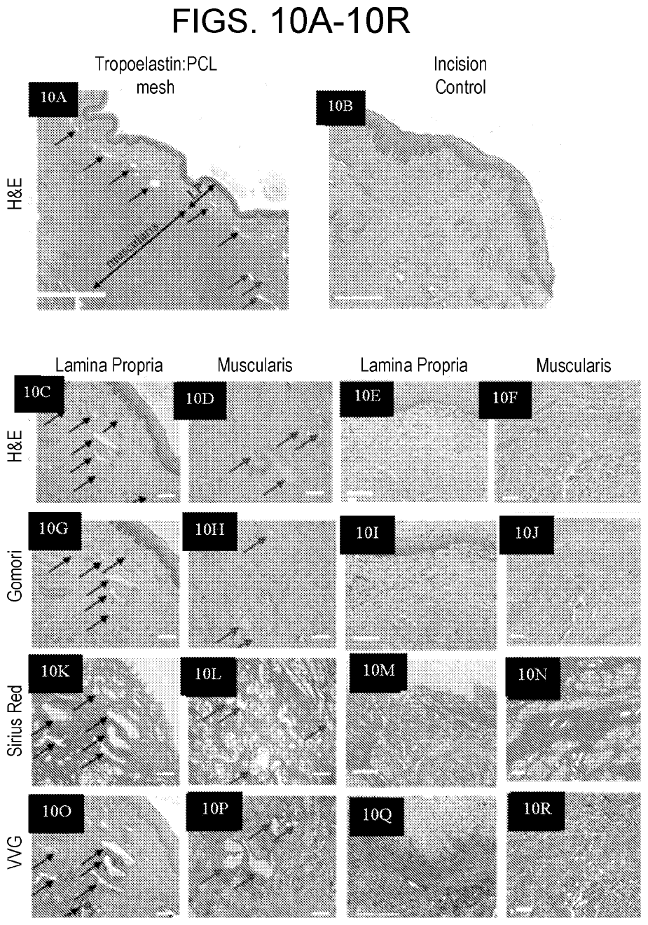

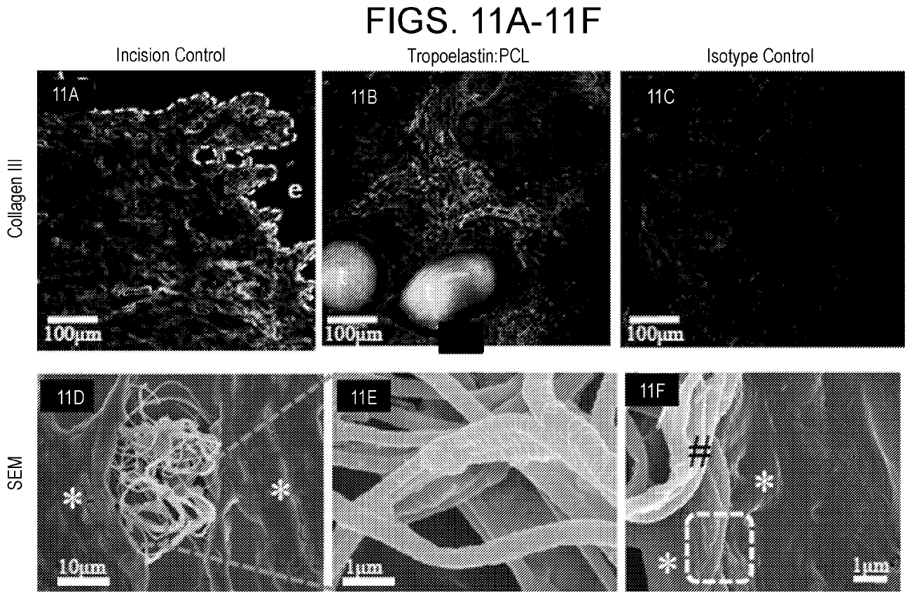

Lack of mesh integration leading to mesh erosion/exposure is one of the major causes of complications associated with commercial polypropylene (PP) meshes. A panoramic image shows the tropoelastin:PCL scaffold was inserted between the lamina propria and muscularis of the ovine vaginal wall (FIGS. 109A and 10C) although some filaments were also in the muscularis (FIG. 10D) after 30 days. In comparison with the incision control (FIGS. 10B, 10E, 10F), the tropoelastin:PCL scaffold appeared to show little disruption to the architecture of the ovine vagina after 30 days in both lamina propria and muscularis. Three connective tissue stains verified the lack of tissue architecture disruption with no scar type collagen evident in Gomori's (FIGS. 10G and 10H) and Sirius red stained sections from the explanted tissue (FIGS. 10K and 10L). The collagen component of both the lamina propria and muscularis appears similar to the incision control (FIGS. 10I, 10J, 10M, 10N). In healing tissue, newly synthesized collagen is characterized by deposition of type III collagen, which appears in greater amount than mature type I collagen as a new ECM matrix is generated to provide support for tissue cells. Collagen III was detected around the tropoelastin:PCL filaments (FIG. 11B) and near the incision (control) (FIG. 11A). These features of appropriate amounts (i.e. not scar like) of collagen III deposition is a hallmark of tissue integration of biomaterial in the host tissue. SEM micrographs also showed evidence of integration with the host tissue (FIGS. 11D, 11F) and confirmed that tropoelastin:PCL scaffolds maintained their structural integrity after 30 days (FIG. 11E). In the Verhoff van Gieson elastin stain, it was apparent that the tropoelastin component of the scaffold was still present after 30 days as it reacted with the stain (FIGS. 11O, 11P). The incision control showed deposition of elastin fibers in the lamina propria around the incision site (FIG. 11Q), confirming the capacity of the injured vagina to synthesizes new elastin fibers. Collectively these results show thorough integration of tropoelastin:PCL electrospun yarn scaffolds in sheep vaginal tissue after 30 days. This is in contrast to Gynemesh®, a discontinued PP mesh used in transvaginal surgery which severely disrupted the muscularis of macaque vagina.

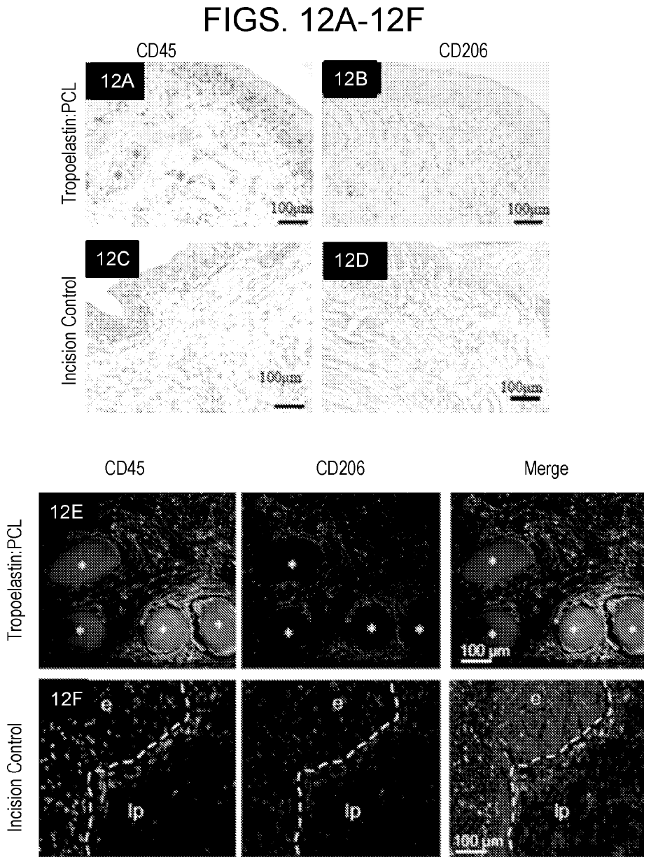

Another important aspect of mesh biocompatibility in vivo is the degree of foreign body response elicited by implanted scaffold biomaterials. Macrophage-mediated foreign body responses to meshes are critical in determining the fate of implanted biomaterial. Our results show similar numbers of CD45+ leukocytes around the elastogen:PCL filaments (FIG. 12A) to that observed in the incision control (FIG. 12C). Similar numbers of M2 wound healing macrophages were also observed in tropoelastin:PCL explanted vagina (FIG. 12B) and the incision control (FIG. 12D). Colocalization studies showed that a substantial proportion of CD45+ leukocytes were CD206+M2 macrophages, particularly at the tropoelastin:PCL filament tissue interface (FIG. 12E). Those CD45+ leukocytes not immunostained with CD206 are likely M0 or M1 inflammatory macrophages. However there are currently no antibodies available that reliably identify M1 macrophages in ovine tissues. These results indicate that the implanted tropoelastin:PCL scaffold elicit a minimal inflammatory response and may even exert an anti-inflammatory effect in vaginal tissue. This minimal foreign body response favors vaginal tissue regeneration. For vaginal application, it is desirable that scaffolds degrade slowly over time as mechanical reinforcement of the pelvic organ support structures is essential for POP repair (ref). While degradable scaffolds may promote integration with host tissue, the dynamic environment and presence of tissue enzymes may cause rapid degradation of material, resulting in treatment failure. Tropoelastin:PCL scaffolds show potential as a suitable implant biomaterial as demonstrated by preliminary results in our ovine POP model.

As indicated in the embodiments above, four different blends of tropoelastin:PCL yarns were successfully fabricated by electrospinning. These yarns were characterized and assessed for their ability to support human dermal fibroblast growth. The results from this study indicate that of the four blends, 50:50 and 25:75 tropoelastin:PCL yarns have characteristics required for use as a scaffold for tissue engineering purposes. 50:50 and 25:75 yarns were consistently capable of being produced in multi-meter lengths, 50:50 yarns released tropoelastin within 7 days, however 25:75 were more structurally stable. Both 50:50 and 25:75 yarns were capable of supporting human dermal fibroblast growth on the surface after 7 days. In an ovine model of POP, transvaginal insertion of the 25:75 tropoelastin:PCL scaffold demonstrated complete integration into the host vaginal tissue eliciting a minimal foreign body response after 30 days. With further research, these scaffolds could be considered as an alternative to non-degradable synthetic nondegradable pelvic organ prolapse mesh products.

1. A method of making a hybrid material, the method comprising:
providing tropoelastin;
providing a biodegradable polymer; and
mixing the tropoelastin and biodegradable polymer to produce a mixture; wherein the mixture results in a hybrid material.

2. The method of claim 1, wherein the biodegradable polymer is polycaprolactone (PCL), poly(lactic acid), poly (lactic-co-glycolic acid, polyglycolic acid, poly(trimethylene carbonate, poly-4-hydroxybutyrate or a co-polymer of any one of the aforementioned polymers.

3. The method of claim 1, wherein the biodegradable polymer is polycaprolactone (PCL).

4. The method of claim 3, wherein the ratio of tropoelastin to PCL is about 75:25, about 50:50 or about 25:75.

5. The method of claim 1, wherein the tropoelastin is provided as a monomer in solution.

6. The method of claim 1, wherein the tropoelastin is provided as tropoelastin particles.

7. The method of claim 1, wherein the method further comprises melting the biodegradable polymer after the providing step, thereby producing a molten biodegradable polymer, and suspending the tropoelastin in the molten biodegradable polymer prior to the mixing step.

8. The method of claim 1, wherein the method further comprises dissolving the biodegradable polymer and dissolving the tropoelastin prior to the mixing step and mixing the dissolved biodegradable polymer and the dissolved tropoelastin.

9. The method of claim 1, wherein the method further comprises dissolving the biodegradable polymer, and suspending the tropoelastin particles in the dissolved biodegradable polymer prior to the mixing step.

10. The method of claim 1, wherein the method further comprises printing or casting the mixture.

11. The method of claim 1, wherein the hybrid material is a yarn.

12. The method of claim 1, wherein the method further comprises electrospinning the mixture, thereby forming an electrospun fibrous yarn.

13. The method of claim 12, wherein the method further comprises collecting the electrospun fibrous yarn.

14. The method of claim 1, wherein the mixture comprises a ratio of tropoelastin to biodegradable polymer of about 99:1, about 95:5, about 90:10, about 80:20, about 70:30, about 75:25, about 60:40, about 50:50, about 40:60, about 30:70, about 25:75, about 10:90 or about 0:100.

15. The method of claim 1, wherein the yarn or electrospun fibrous yarn comprises a length of about 1 cm, about 5 cm, about 15 cm, 15 cm, about 20 cm, about 25 cm, about 30 cm, about 35 cm, about 40 cm, about 45 cm, about 50 cm, about 75 cm, about 100 cm, about 125 cm, about 150 cm, about 175 cm, about 200 cm, about 225 cm, about 250 cm, about 275 cm, about 300 cm, about 325 cm, about 350 cm, about 375 cm, about 400 cm, about 425 cm, about 450 cm, about 475 cm, about 500 cm, about 525 cm, about 550 cm, about 575 cm, about 600 cm, about 625 cm, about 650 cm, about 675 cm, about 700 cm or any length in between a range defined by any two aforementioned values.

16. A hybrid material, the material comprising:
tropoelastin; and
a biodegradable polymer.

17. The hybrid material of claim 17, wherein the hybrid material is a yarn.

18. The hybrid material of claim 17, wherein the biodegradable polymer is polycaprolactone (PCL), poly(lactic acid), poly (lactic-co-glycolic acid, polyglycolic acid, poly(trimethylene carbonate, poly-4-hydroxybutyrate or a co-polymer of any one of the aforementioned polymers.

19. A method of tissue repair, the method comprising:
providing a tissue engineering scaffold, wherein the tissue engineering scaffold comprises a hybrid yarn, the yarn comprising:
tropoelastin; and
a biodegradable polymer; and
implanting the tissue engineering scaffold into tissue of an individual.

20. The method of claim 19, wherein the method promotes deposition of collagen into the tissue of the individual.
